SEQUENCE LISTING (18 sequences)

SEQ ID NO: 1 - DNA, 3498 bp, Homo sapiens
  CDS (266)..(2623)
ttctttccaagggtctctgggtgaggcccgtgaccttcccaagcctctccctgtcttgtgaaacctgggcgtgatatacctcccttttagggctgctgcgatcatttaggcagattaaacctcataagtggtttcccatacaagaaagatgctagcagtgcaacagacagaacacttacctgcctgccctcccgccaggaggtggtcttccaacttttgcccggagtctacagagggtgggccctctctgctggggctccgggacatggtcaggagaggttggtctgtctgtaccgccattctcttggccagactgtggtgtctggtccctactcacaccttcctgtcagagtatccagaggccgcagagtatccacaccctggctgggtgtactggctacagatggctgtggctccaggtcacctgcgtgcctgggtgatgagaaataatgtcacaacaaatatcccatctgcattctctgggacactgacccatgaagagaaagcagttctcacagtttttacaggcacagccacagccgtgcatgtacaggtggcagctttagcttctgctaaactggagagctcagtgtttgtgacagactgcgtgtcctgcaaaatcgaaaatgtctgtgattcagctcttcagggaaaaagggtgccgatgtctggcctacagggctcaagcattgtcatcatgcccccatccaaccgtccactcgccagtgcggcatcctgcacgtggtcagtccaagtccagggagggccccatcacctgggggtggtcgctatcagtggcaaagtcttgtcagcagctcatggggcaggaagggcctatggttgggggtttcctggcgatcccatggaggaaggatacaagaccctcctgaaaggaatttccgggaagttcaatagtggtgagttggtggccattatgggtccttccggggccgggaagtccacgctgatgaacatcctggctggatacagggagacgggcatgaagggggccgtcctcatcaacggcctgccccgggacctgcgctgcttccggaaggtgtcctgctacatcatgcaggatgacatgctgctgccgcatctcactgtgcaggaggccatgatggtgtcggcacatctgaagcttcaggagaaggatgaaggcagaagggaaatggtcaaggagatactgacagcgctgggcttgctgtcttgcgccaacacgcggaccgggagcctgtcaggtggtcagcgcaagcgcctggccatcgcgctggagctggtgaacaaccctccagtcatgttcttcgatgagcccaccagcggcctggacagcgcctcctgcttccaggtggtctcgctgatgaaagggctcgctcaagggggtcgctccatcatttgcaccatccaccagcccagcgccaaactcttcgagctgttcgaccagctttacgtcctgagtcaaggacaatgtgtgtaccggggaaaagtctgcaatcttgtgccatatttgagggatttgggtctgaactgcccaacctaccacaacccagcagattttgtcatggaggttgcatccggcgagtacggtgatcagaacagtcggctggtgagagcggttcgggagggcatgtgtgactcagaccacaagagagacctcgggggtgatgccgaggtgaacccttttctttggcaccgcccctctgaagaggtaaagcagacaaaacgattaaaggggttgagaaaggactcctcgtccatggaaggctgccacagcttctctgccagctgcctcacgcagttctgcatcctcttcaagaggaccttcctcagcatcatgagggactcggtcctgacacacctgcgcatcacctcgcacattgggatcggcctcctcattggcctgctgtacttggggatcgggaacgaaaccaagaaggtcttgagcaactccggcttcctcttcttctccatgctgttcctcatgttcgcggccctcatgcctactgttctgacatttcccctggagatgggagtctttcttcgggaacacctgaactactggtacagcctgaaggcctactacctggccaagaccatggcagacgtgccctttcagatcatgttcccagtggcctactgcagcatcgtgtactggatgacgtcgcagccgtccgacgccgtgcgctttgtgctgtttgccgcgctgggcaccatgacctccctggtggcacagtccctgggcctgctgatcggagccgcctccacgtccctgcaggtggccactttcgtgggcccagtgacagccatcccggtgctcctgttctcggggttcttcgtcagcttcgacaccatccccacgtacctacagtggatgtcctacatctcctatgtcaggtatgggttcgaaggggtcatcctctccatctatggcttagaccgggaagatctgcactgtgacatcgacgagacgtgccacttccagaagtcggaggccatcctgcgggagctggacgtggaaaatgccaagctgtacctggacttcatcgtactcgggattttcttcatctccctccgcctcattgcctatttggtcctcaggtacaaaatccgggcagagaggtaaaacacctgaatgccaggaaacaggaagattagacactgtggccgagggcacgtctagaatcgaggaggcaagcctgtgcccgaccgacgacacagagactcttctgatccaacccctagaaccgcgttgggtttgtgggtgtctcgtgctcagccactctgcccagctgggttggatcttctctccattcccctttctagctttaactaggaagatgtaggcagattggtggttttttttttttttttaacatacagaattttaaataccacaactggggcagaatttaaagctgcaacacagctggtgatgagaggcttcctcagtccagtcgctccttagcaccaggcaccgtgggtcctggatggggaactgcaagcagcctctcagctgatggctgcacagtcagatgtctggtggcagagagtccgagcatggagcgattccattttatgactgttgtttttcacattttcatctttctaaggtgtgtctcttttccaatgagaagtcatttttgcaagccaaaagtcgatcaatcgcattcattttaagaaattatacctttttagtacttgctgaagaatgattcagggtaaatcacatactttgtttagagaggcgaggggtttaacccgagtcacccagctggtctcatacatagacagcacttgtgaaggattgaatgcaggttccaggtggagggaagacgtggacaccatctccactgagccatgcagacatttttaaaagctatacacaaaattgtgagaagacattggccaactctttcaaagtctttctttttccacgtgcttcttattttaagcgaaatatattgtttgtttcttcctaaaaaaaaaaaaaaaaaaaaa

SEQ ID NO: 2 - protein, 785 aa, Homo sapiens [sequence omitted]

SEQ ID NO: 3 - RNA, 21 nt, Artificial sequence
  siRNA (forward strand)
ucauuggccugcuguacuuuu

SEQ ID NO: 4 - RNA, 21 nt, Artificial sequence
  siRNA (reverse strand)
aaguacagcaggccaaugauu

SEQ ID NO: 5 - RNA, 21 nt, Artificial sequence
  siRNA (forward strand)
gcgcaucaccucgcacauuuu

SEQ ID NO: 6 - RNA, 21 nt, Artificial sequence
  siRNA (reverse strand)
aaugugcgaggugaugcgcuu

SEQ ID NO: 7 - RNA, 21 nt, Artificial sequence
  siRNA (forward strand)
ggaaauggucaaggagauauu

SEQ ID NO: 8 - RNA, 21 nt, Artificial sequence
  siRNA (reverse strand)
uaucuccuugaccauuuccuu

SEQ ID NO: 9 - RNA, 21 nt, Artificial sequence
  siRNA (forward strand)
ggaaauggucaaggagauauu

SEQ ID NO: 10 - RNA, 21 nt, Artificial sequence
  siRNA (reverse strand)
uuucaggagggucuuguauuu

SEQ ID NO: 11 - DNA, 601 bp, Homo sapiens
  allele (301)..(301): k=G/T
cttcaccagctcactttcccccactttgctgcaataatcattggctagaggtattgtgatatgatgtcattaaagttaatctagacgaaaatttgatttacctaaaactattacactgtagacctggaggaatttcagtttttgccgtaattgttttcaatgtgtgttataaaaaataaattccactatgttcacgaatgtacaacttataatctgaccaaaagtgagagatgggtagattttcctacttgggtccttctgtggacaggtactaggtgctgctttacgcccagtgacttgkgagggaacagaactgccctttagtaaccctgctcacttcctgttttctgcagtaacaattacattaggcaatattattacacatggaattgcatcatgctgattttaaaacaatcctccctgtagcattacacagacactgaatcatcatttgtagtttggggggctttacatgcctgcagtggtgaaaactgaaattttgtcccacttaagggagtttcttcttccctttattaattgcaaaataaatatatgtcacttcagagggcagcagctggactacctatgtttgtgggtaagt

SEQ ID NO: 12 - DNA, 601 bp, Homo sapiens
  allele (301)..(301): m=A/C
agccttcattccattcgtccttgttaccaggtttctgctaagctcccttccagggctgagatctcagaggcttcaccagctcactttcccccactttgctgcaataatcattggctagaggtattgtgatatgatgtcattaaagttaatctagacgaaaatttgatttacctaaaactattacactgtagacctggaggaatttcagtttttgccgtaattgttttcaatgtgtgttataaaaaataaattccactatgttcacgaatgtacaacttataatctgaccaaaagtgagagmtgggtagattttcctacttgggtccttctgtggacaggtactaggtgctgctttacgcccagtgacttgtgagggaacagaactgccctttagtaaccctgctcacttcctgttttctgcagtaacaattacattaggcaatattattacacatggaattgcatcatgctgattttaaaacaatcctccctgtagcattacacagacactgaatcatcatttgtagtttggggggctttacatgcctgcagtggtgaaaactgaaattttgtcccacttaagggagtttcttcttccctt

SEQ ID NO: 13 - DNA, 20 bp, Artificial sequence
  primer
gcttcaccagctcactttcc

SEQ ID NO: 14 - DNA, 21 bp, Artificial sequence
  primer
catgatgcaattccatgtgta

SEQ ID NO: 15 - RNA, 21 nt, Artificial sequence
  siRNA (forward strand)
gcgaagcuguaccuggauuuu

SEQ ID NO: 16 - RNA, 21 nt, Artificial sequence
  siRNA (reverse strand)
aauccagguacagcuucgcuu

SEQ ID NO: 17 - DNA, 19 bp, Artificial sequence
  primer
cgtgcatgaatcacaaaaa

SEQ ID NO: 18 - DNA, 19 bp, Artificial sequence
  primer
caccactgcaggcatgtaa

(19) United States
(12) Patent Application Publication
(10) Pub. No.: US 2013/0289099 A1
(43) Pub. Date: Oct. 31, 2013

(54) ABCG1 GENE AS A MARKER AND A TARGET GENE FOR TREATING OBESITY

(71) Applicants: Wilfried Le Goff, Versailles (FR); Maryline Olivier, Villepreux (FR); Maryse Guerin, Paris (FR)

(72) Inventors: Wilfried Le Goff, Versailles (FR); Maryline Olivier, Villepreux (FR); Maryse Guerin, Paris (FR)

(73) Assignee: UNIVERSITE PIERRE ET MARIE CURIE (PARIS 6), Paris (FR)

(21) Appl. No.: 13/993,726

(22) PCT Filed: Dec. 16, 2011
(86) PCT No.: PCT/EP2011/073140

§ 371 Date: Jun. 13, 2013

(30) Foreign Application Priority Data
Dec. 17, 2010 (EP) 10306449.9

(51) Int. Cl. C12N 15/113

(52) U.S. Cl. CPC C12N 15/1138
USPC 514/44 A; 435/6.11

(57) ABSTRACT

The invention relates to a method for treating obesity in a patient, which method comprises administering an effective quantity of ABCG1 inhibitor to a patient in need thereof. The invention further provides an in vitro method for determining whether a patient is at risk of developing obesity, which method comprises detecting the presence of a mutation, substitution or deletion of at least one nucleotide in ABCG1 20 gene or regulatory sequences thereof.

The invention relates to the use of ATP-binding cassette G1 (ABCG1) gene as a target gene for treating obesity, and as a marker for diagnosing a higher risk of developing obesity.

### TECHNICAL BACKGROUND

The recent rise in the prevalence of obesity is an issue of major concern for the health systems of several countries.

Obesity is often defined simply as a condition of abnormal or excessive fat accumulation in adipose tissue, to the extent that health may be impaired. The underlying disease is the process of undesirable positive energy balance and weight gain. An abdominal fat distribution is associated with higher health risks than a gynoid fat distribution.

Potentially life-threatening, chronic health problems associated with obesity fall into four main areas: 1) cardiovascular problems, including hypertension, chronic heart disease and stroke, 2) conditions associated with insulin resistance, namely Non-Insulin Dependent Diabetes Mellitus (NIDDM), 3) certain types of cancers, mainly the hormonally related and large-bowel cancers, and 4) gallbladder disease. Other problems associated with obesity include respiratory difficulties, chronic musculo-skeletal problems, skin problems and infertility.

The main currently available strategies for treating these disorders include dietary restriction, increments in physical activity, pharmacological and surgical approaches. In adults, long term weight loss is exceptional using conservative interventions. Present pharmacological interventions typically induce a weight loss of between five and fifteen kilograms; if the medication is discontinued, renewed weight gain ensues. Surgical treatments are comparatively successful and are reserved for patients with extreme obesity and/or with serious medical complications.

The ATP-binding cassette G1 (ABCG1) membrane transporter was shown to play a key role in cellular lipid homeostasis in mice by mediating cellular free cholesterol efflux to high-density lipoproteins (HDL) (Wang et al., 2004), a major step in the reverse cholesterol transport pathway. Neutral lipid accumulation was observed in the lungs of Abcg1 KO mice when fed a normal chow diet (Kennedy et al., 2005). In addition, Abcg1 KO mice failed to maintain cellular lipid homeostasis in both hepatocytes and in tissue macrophages following administration of a high-fat/high-cholesterol diet. Significantly, the expression of the ABCG1 transporter is strongly induced upon cellular sterol loading (Baldan et al., 2009). Furthermore ABCG1 is expressed in adipocytes and in adipose tissue of mice which develop diet-induced obesity (Buchmann et al., 2007).

To date, the physiological function of ABCG1 in lipid metabolism in humans is indeterminate. Indeed, no genetic diseases caused by ABCG1 mutations have been described in man, and no association between ABCG1 single nucleotide polymorphisms (SNPs) and human pathologies have been identified by genome-wide association studies (GWAS) (www.genome.gov/gwastudies) (Hindorff et al., 2009). So far, only observational data have been reported (Mauldin et al., 2008; Thomassen et al., 2007). ABCG1 expression is correlated with cholesterol accumulation in macrophages from patients with type 2 diabetes mellitus (Mauldin et al., 2008). Furthermore a recent study in patients with severe pulmonary alveolar proteinosis with decreased ABCG1 expression levels and lipid accumulation in pulmonary macrophages suggested a role for ABCG1 in surfactant homeostasis (Thomassen et al., 2007). However no causal relationship was demonstrated in these studies. Finally, several polymorphisms have been reported in the human ABCG1 gene (lida et al., 2002), and associations between ABCG1 SNPs and neuropsychiatric disorders and behavioral traits have been documented (Kirov et al., 2001; Nakamura et al., 1999)

### SUMMARY OF THE INVENTION

It is now described a method for treating obesity in a patient, which method comprises administering an effective quantity of an inhibitor of ABCG1 to a patient in need thereof.

The invention thus provides an inhibitor of ATP-binding cassette G1 (ABCG1) gene expression or activity, for use in treating obesity, preferably morbid or abdominal obesity, in a patient.

It is also herein described the use of an inhibitor of ABCG1, for the preparation of a medicament for treating obesity in a patient.

In a preferred embodiment, the inhibitor is a nucleic acid, such as a siRNA or an antisense nucleic acid. Preferably the inhibitor is a siRNA that comprises a nucleotide sequence selected from SEQ ID NO: 3 to SEQ ID NO:10.

In a preferred embodiment, the inhibitor of ABCG1 is a nucleic acid carried by an expression vector, such as a virus vector, preferably an adenovirus vector.

In a most preferred embodiment, the ABCG1 inhibitor is a siRNA that represses ABCG1 expression, particularly useful in treating obesity by injection in the abdomen patient.

The invention further provides an in vitro method for determining whether a patient is at risk of developing obesity, which method comprises detecting the presence of a mutation, substitution or deletion of at least one nucleotide in ABCG1 gene or regulatory sequences thereof, preferably in the promoter of the gene.

### LEGENDS TO THE FIGURES

FIGS. 1A to 1D show that suppression of ABCG1 expression leads to a reduced lipoprotein lipase (LPL) activity. FIG. 1A is a graph that shows LPL activity measured in pre- and postheparin plasmas (100 U/Kg) from wild-type (WT) and Abcg1 knockout (KO) mice following an overnight fast. 1 mU represents 1 nmol of Free Fatty Acid released per minute. n=5 mice per group. *p<0.01. FIG. 1B is a histogram that shows relative quantification of LPL mRNA levels in freshly isolated human monocytes (D0) from blood samples and following a 12-day differentiation period into human macrophages (D12). LPL mRNA levels were normalized to housekeeping genes (8-aminolevulinate synthase, hypoxanthine phosphoribosyltransferase and α-tubulin). FIG. 1C is a Western blot assessing total ABCG1 protein in human monocyte-derived macrophages (HMDM) transfected with control siRNA (Ctrl) or siRNA targeting human ABCG1 (ABCG1 Knockdown, KD). FIG. 1D is a histogram that shows LPL activity measured in culture media from control (Ctrl) and ABCG1 KD HMDM. Values are means±SEM of 3 independent experiments performed in triplicate. *p<0.05.

FIG. 2 is a graph that shows that LPL expression is elevated at the cell surface of ABCG1 KD human macrophages relative to control cells. Cell surface LPL was quantified in Ctrl and ABCG1 KD THP-1 macrophages by flow cytometry. Values are means±SEM of 5 independent experiments performed in duplicate. *p<0.05.

FIGS. 3A to 3C show that ABCG1 promotes LPL-mediated lipid accumulation from VLDL in human macrophages. Cellular triglyceride (FIG. 3A) and cholesterol (FIG. 3B) contents were measured in Ctrl and ABCG1 KD HMDM after a 24 h-incubation with 50 μg/mL VLDL with or without LPL inhibitor (THL). FIG. 3C, Relative mRNA levels in Ctrl and ABCG1 KD HMDM normalized to housekeeping genes (8-aminolevulinate synthase, hypoxanthine phosphoribosyltransferase and α-tubulin). Values are means±SEM of 3 independent experiments performed in triplicate *p<0.01 and **p<0.0001.

FIGS. 4A to 4C are graphs that show that inhibition of ABCG1 expression reduces triglyceride (TG) storage in adipocytes. In FIG. 4A, efficiency of the ABCG1 knockdown in 3T3-L1 adipocytes was assessed by quantification of mRNA. ABCG1 mRNA was normalized to housekeeping genes (hypoxanthine phosphoribosyltransferase and cyclophilin A). In FIG. 4B, secreted LPL activity was measured in the culture media of Ctrl and ABCG1 KD 3T3-L1 adipocyte. In FIG. 4C, cellular triglyceride content was quantified during maturation of 3T3-L1 preadipocytes into adipocytes nucleofected with control siRNA (Ctrl) or siRNA targeting ABCG1 (ABCG1 KD). Values are means±SEM of 5 independent experiments performed in duplicate *p<0.05 and **p<0.0005.

FIGS. 5A and 5B are graphs that show that the AT haplotype is associated with higher BMI in obese individuals and with increased ABCG1 promoter activity. FIG. 5A shows the amount of −206A/−136T (AT) haplotypes relative to BMI in obese individuals. AT/AT=2. FIG. 5B shows human ABCG1 promoter activity in relation to the CG and AT haplotypes. HepG2 cells were transiently transfected with a construct containing the proximal 1056 bp of the human promoter with either the −206A/−136T (AT) haplotype or the −206C/−136G (CG) haplotype. Luciferase activity is expressed in RLU (Relative Lucifersase Unit) after normalization for β-galactosidase activity. Values are means±SEM of 5 independent experiments performed in triplicate. *p<0.0005.

FIGS. 6A to 6F are graphs that show the analysis of the −134T/G and −204A/C ABCG1 SNPs in a large cohort of obese patients. Association of the −134T/G (FIGS. 6A, 6C, 6E) and −204A/C (FIGS. 6B, 6D) ABCG1 SNP with BMI (FIGS. 6A-6B), fat mass index (FMI, FIG. 6C) and adiponectin levels (FIGS. 6D, 6E). FIG. 6F shows the amount of −204A/−134T (AT) haplotypes relative to BMI in obese individuals. AT/AT=2. The effect of each SNP on BMI was analyzed by linear regression in an additive, dominant and recessive manner. All models were adjusted for age and sex.

FIGS. 7A to 7B are graphs that show Elevated ABCG1 expression and adipocyte diameter in adipose tissue from individuals carrying the AT haplotype. FIG. 7A shows BMI in 10 obeses individuals carrying either the AT or the GC haplotype. In FIG. 7B, mRNA were isolated from adipose tissue biopsies and ABCG1 mRNA levels were normalized to human non-POU domain containing, octamer-binding housekeeping gene (NONO), human α-tubulin (TUBA) and human heat shock protein 90 kDa alpha (cytosolic), class B member 1 (HSP90AB1). *p<0.05 and **p<0.0001 versus GC haplotype. FIG. 7C shows the correlation between adipocyte diameter and ABCG1 expression in adipose tissue from obese patients. n=20.

FIGS. 8A to 8D are graphs that show increased expression of markers specific to adipocyte differentiation, maturation and inflammations in adipose tissue from individuals carrying the AT haplotype. mRNAs were isolated in adipose tissue biopsies from 10 individuals carrying either the AT or the GC haplotype. PPARy (FIG. 8A), perilipin (FIG. 8B), CD36 (FIG. 8C) and TNFα (FIG. 8D) mRNA levels were normalized to human non-POU domain containing, octamer-binding housekeeping gene (NONO), human α-tubulin (TUBA) and human heat shock protein 90 kDa alpha (cytosolic), class B member 1 (HSP90AB1). *p<0.05 versus GC haplotype.

FIGS. 9A to 9C are graphs that show that local delivery of lentiviral particles inhibiting ABCG1 expression by RNAi in adipose tissue led to a marked reduction of weight gain in mice. C57BL/6 mice fed a high fat diet (40% fat) were injected locally in the epididymal adipose tissue with lentiviral particles encoding either a shRNA inhibiting mouse ABCG1 expression (lenti-ABCG1) or a shRNA control (lenti-Ctrl). Weight gain in mice (FIG. 9A), mRNA levels (FIG. 9B) of ABCG1 and adipocyte diameter in epididymal adipose tissue (FIG. 9C) were calculated after 4 weeks following the day of the injection. n=10 mice per group. *p<0.05 versus lenti-Ctrl. Values are the mean±SEM of two independent experiments.

### DETAILED DESCRIPTION OF THE INVENTION

The inventors have identified a major role for ABCG1 in human pathophysiology. Indeed, the inventors have shown that ABCG1 promotes cellular TG accumulation and thus contributes to human macrophage foam cell formation and adipocyte TG storage. Moreover, they have demonstrated that ABCG1 is associated with obesity and report the interest of inhibiting ABCG1 to treat individuals developing obesity, especially abdominal obesity.

### DEFINITIONS

The ABCG1 gene encodes the ATP-binding cassette, subfamily G member 1. It was mapped to chromosome 21q22.3. Langmann et al. (2000) determined that the ABCG1 gene spans more than 70 kb and contains 15 exons that range in size from 51 to 1081 bp. Using promoter luciferase reporter analysis, they found that the first exon extends 110 bp upstream from the ATG start codon and that the proximal 5-prime flanking region contains the TATA-less, GC-rich ABCG1 promoter. Transient transfection experiments showed that the promoter region contains silencing elements that can mediate functional transcriptional repression. Using RACE assays, Lorkowski et al. (2001) determined that the ABCG1 gene contains 5 exons more that what was previously reported, 4 upstream and 1 downstream of the previous exon 1, and spans 97 kb. The novel exons are predicted to encode at least 5 novel transcripts. Additional promoter regions were identified upstream of exons 1 and 5, respectively. A human ABCG1 gene sequence (mRNA, with complete coding sequence, alternatively spliced) is shown as SEQ ID NO:1, and the corresponding protein sequence is shown as SEQ ID NO:2.

The term “ABCG1 function” or “ABCG1 activity” includes cholesterol and phospholipid transport, especially in macrophages. According to the present invention, it further includes cellular TG accumulation, and adipocyte TG storage.

Obesity is defined as a condition of abnormal or excessive accumulation of adipose tissue. “Morbid obesity” refers to severe obesity which may lead to health impairment. The body mass index (BMI; kg/m²) provides the most useful, albeit crude, population-level measure of obesity. Obesity has also been defined using the WHO classification of the BMI classes for adults: underweight (<18.5), normal weight (18.5 to 24.99), overweight (25 to 29.99), obese grade I (30 to 34.99), obese grade II (35 to 39.99), obese grade III and more (40). See WHO, Global database on Body Mass Index.

Abdominal obesity, also designated as central obesity, is the accumulation of abdominal or visceral fat resulting in an increase in waist size. There is a strong correlation between central obesity and cardiovascular disease. Visceral fat, also known as organ fat or intra-abdominal fat, is located inside the peritoneal cavity, packed in between internal organs and torso, as opposed to subcutaneous fat which is found underneath the skin, and intramuscular fat which is found interspersed in skeletal muscle. Visceral fat is composed of several adipose depots including mesenteric, epididymal white adipose tissue (EWAT) and perirenal fat. While central obesity can be obvious just by looking at the naked body, the severity of central obesity is determined by taking waist and hip measurements. The absolute waist circumference (>102 centimetres in men and >88 centimetres in women) and the waist-hip ratio (>0.9 for men and >0.85 for women) are both used as measures of central obesity.

The term “inhibitor of ABCG1” as used herein means a substance that decreases the level of expression or activity of ABCG1 protein in a cell, by modification of the levels and/or activity of the protein, or by modification of the level of ABCG1 gene transcription. Inhibitors can be compounds that block, antagonize, prevent, or reduce the activity of ABCG1. Nucleic acid molecules capable of mediating RNA Interference (RNAi), such as siRNA, antisense nucleic acids, as well as small molecule inhibitors directed to ABCG1 can be potential inhibitors of ABCG1 activity.

The term “RNAi” as used herein means RNA interference process for a sequence-specific post-transcriptional gene silencing or gene knockdown by providing a double-stranded RNA (dsRNA) that is homologous in sequence to the targeted gene. Small interfering RNAs (siRNAs) can be synthesized in vitro or generated by ribonuclease III cleavage from longer dsRNA and are the mediators of sequence-specific mRNA degradation. The currently known mechanism of RNAi can be described as follows: The processing of dsRNA into siRNAs, which in turn induces degradation of the intended target mRNA, is a two-step RNA degradation process. The first step involves a dsRNA endonuclease (ribonuclease III-like; RNase III-like) activity that processes dsRNA into smaller sense and antisense RNAs which are most often in the range of 21 to 25 nucleotides (nt) long, giving rise to the so called short interfering RNAs (siRNAs). This RNase III-type protein is termed “Dicer”. In a second step, the antisense siRNAs produced combine with, and serve as guides for, a different ribonuclease complex called RNA-induced silencing complex (RISC), which allows annealing of the siRNA and the homologous single-stranded target mRNA, and the cleavage of the target homologous single-stranded mRNAs. Cleavage of the target mRNA has been observed to place in the middle of the duplex region complementary to the antisense strand of the siRNA duplex and the intended target mRNA. Micro RNAs (miRNAs) constitute non coding RNAs of 21 to 25 nucleotides, which controls genes expression at post-transcriptional level. miRNAs are synthesized from ARN polymerase II or ARN polymerase III in a pre-miRna of 125 nucleotides. Pre-miRNA are cleaved in the nucleus by the enzyme Drosha, giving rise to a precursor called imperfect duplex hairpin RNA (or miRNA-based hairpin RNA). These imperfect duplex hairpin RNAs are exported from the nucleus to the cytoplasm by exportin-5 protein, where it is cleaved by the enzyme DICER, giving rise to mature miRNAs. miRNAs combine with RISC complex which allows total or partial annealing with the homologous single-stranded target mRNA. Partial annealing with the mRNA leads to the repression of protein translation, whereas total annealing leads to cleavage of the single-stranded mRNA.

“An antisense nucleic acid” refers to a nucleic acid comprising a nucleotide sequence hybridizable specifically with a target mRNA (mature mRNA or initial transcription product) under physiological conditions for the cells that express the target mRNA, and being capable of inhibiting the translation of the polypeptide encoded by the target mRNA in a hybridized state. The choice of antisense nucleic acid may be a DNA or an RNA, or a DNA/RNA chimera, and is preferably a DNA.

The term “specifically hybridize” as used herein means that under appropriate conditions a probe made or a nucleic acid sequence such as an siRNA oligo hybridizes, duplexes or binds only to a particular target DNA or RNA sequence present in a cell or preparation of DNA or RNA. A probe sequence such as an siRNA sequence specifically hybridizes to a target sequence when the base sequence of the probe nucleic acid and the target sequence are complimentary to one another. The target sequence and the probe sequence do not have to be exactly complimentary to one another in order for the probe sequence to specifically hybridize. It is understood that specific hybridization can occur when the target and probe sequences are not exactly complimentary to one another and specific hybridization can occur when up only about 80% of the bases are complimentary to one another. Preferably, it is understood that in specific hybridizations probe and target sequence have 80% comprehensibility to one another. For discussions on hybridization see for example, Current Protocols in Molecular Biology, F. Ausubel et al., (ed.) Greene Publishing and Wiley-Interscience, New York (July, 2002).

The term “treating” as used herein means the prevention, reduction, partial or complete alleviation or cure of a disease.

The term “patient” or “subject” means any mammal, preferably a human being, of any age or sex. Preferably adults or adolescents are advantageously treated according to the invention.

### Therapeutic Methods

### Inhibitors of ABCG1:

The present invention provides a treatment of obesity, by inhibiting expression or activity of ABCG1.

In a preferred embodiment, it advantageously employs RNA interference, especially siRNA oligonucleotides directed to ABCG1, which specifically hybridize nucleic acids encoding ABCG1 and interfere with ABCG1 gene expression. Accordingly ABCG1 proteins levels are reduced and the total level of ABCG1 activity in the cell is reduced.

Using the present invention it is possible to observe the function of ABCG1. In addition, specific siRNA oligos directed to ABCG1 have been designed and tested in human cells showing a reduction in secreted LPL activity by trapping LPL protein at the cell surface with their use. These siRNA and equivalent compounds may have therapeutic value in the treatment of obesity as described herein. It is therefore understood that compounds that inhibit ABCG1 expression and/or ABCG1 protein activity also have therapeutic value.

Various means for RNA interference may be used. The present invention relates to compounds, compositions, and methods useful for modulating the expression and activity of ABCG1 by RNA interference (RNAi) using small nucleic acid molecules, such as micro RNA (miRNA), short-hairpin RNA (shRNA) and/or short or small interfering RNA (siRNA).

Preferably the siRNA is used in form of synthetic RNA duplexes (ds-siRNAs), i.e, the siRNA is a siRNA duplex comprised of a sense strand homologue to the target and an antisense strand that binds to the target mRN). However single stranded siRNAs (ss-siRNA) was be of use also.

The length of the portion complementary to the target nucleotide sequence, contained in the siRNA, is generally about 18 bases or more, preferably 19 bases or more, more preferably about 21 bases or more, but is not limited, as far as the expression of the target gene can specifically be suppressed. If the siRNA is longer than 23 bases, the siRNA may undergo degradation in cells to produce an siRNA having about 20 bases in length; therefore, theoretically, the upper limit of the portion complementary to the target nucleotide sequence is the full length of the nucleotide sequence of an mRNA (mature mRNA or initial transcription product) of the target gene. Taking into account the avoidance of interferon induction, the ease of synthesis, antigenicity issues and the like, however, the length of the complementary portion is, for example, about 50 bases or less, preferably about 25 bases or less, most preferably about 23 bases or less. Hence, the length of the complementary portion is generally about 18 to 50 bases, preferably about 19 to about 25 bases, more preferably about 21 to about 23 bases.

The length of each RNA strand that constitutes the siRNA is generally about 18 bases or more, preferably 19 bases or more, more preferably about 21 bases or more, but is not limited, as far as the expression of the target gene can specifically be suppressed; there is theoretically no upper limit on the length of each RNA strand. Taking into account the avoidance of interferon induction, the ease of synthesis, antigenicity issues and the like, however, the length of the siRNA is, for example, about 50 bases or less, preferably about 25 bases or less, most preferably about 23 bases or less. Hence, the length of each RNA strand is, for example, generally about 18 to 50 bases, preferably about 19 to about 25 bases, more preferably about 21 to about 23 bases. The length of the shRNA is expressed as the length of the double-stranded moiety when the shRNA assumes a double-stranded structure.

It is preferable that the target nucleotide sequence and the sequence complementary thereto contained in the siRNA be completely complementary to each other. However, in the presence of a base mutation at a position apart from the center of the siRNA, the cleavage activity by RNA interference is not completely lost, but a partial activity can remain. On the other hand, a base mutation in the center of the siRNA has a major influence to the extent that it can extremely reduce the mRNA cleavage activity by RNA interference.

The siRNA may have an additional base that does not form a base pair at the 5′- and/or 3′-terminal. The length of the additional base is not particularly limited, as far as the siRNA can specifically suppress the expression of the target gene; the length is generally 5 bases or less, for example, 2 to 4 bases. Although the additional base may be a DNA or an RNA, use of a DNA improves the stability of the siRNA. Examples of the sequences of such additional bases include, but are not limited to, the sequences ug-3′, uu-3′, tg-3′, tt-3′, ggg-3′, guuu-3′, gttt-3′, ttttt-3′, uuuuu-3′ and the like.

Preferred molecules capable of mediating RNA interference advantageously down regulate at least 60%, preferably at least 70%, preferably at least 80%, even more preferably at least 90%, of the target protein expression.

siRNA oligonucleotides designed to silence ABCG1 gene are commercially available, e.g. from Dharmacon or Santa Cruz Biotechnoloy, Ambion, Abnova, Sigma-Aldrich, Invitrogen—Life Technologies, Qiagen, Applied Biosystems—Life Technologies, Eurofins MWG/operon, Origene,

Preferred siRNA designed to silence the human ABCG1 gene are identified below:

1- Forward
(SEQ ID NO: 3)
5′-UCAUUGGCCUGCUGUACUU-UU-3′
1- Reverse
(SEQ ID NO: 4
5′-P-AAGUACAGCAGGCCAAUGA-UU-3′
2- Forward
(SEQ ID NO: 5)
5′-GCGCAUCACCUCGCACAUU-UU-3′
2- Reverse
(SEQ ID NO: 6)
5′-P-AAUGUGCGAGGUGAUGCGC-UU-3′
3- Forward
(SEQ ID NO: 7)
5′-GGAAAUGGUCAAGGAGAUA-UU-3′
3- Reverse
(SEQ ID NO: 8)
5′-P-UAUCUCCUUGACCAUUUCC-UU-3′
4- Forward
(SEQ ID NO: 9)
5′-GGAAAUGGUCAAGGAGAUA-UU-3′
4- Reverse
(SEQ ID NO: 10)
5′-P-UUUCAGGAGGGUCUUGUAU-UU-3′

In a preferred embodiment, the invention makes use of a siRNA that shows a nucleotide sequence selected from the group consisting of SEQ ID NO: 3 to SEQ ID NO:10, preferably in duplex form.

The above described siRNA molecules may be either synthesized or produced by cleavage of corresponding shRNAs by DICER. Such shRNAs can be produced from vectors comprising corresponding nucleic acid sequences.

Other siRNA sequences that silence ABCG1 can be easily designed by any person skilled in the art.

Without intending to be limited by mechanism, it is believed that an ABCG1 specific inhibitor acts by reducing the amount of activity of ABCG1 protein and/or ABCG1 expression in a cell, thereby directly or indirectly reducing the secreted LPL activity, by trapping LPL protein at the cell surface.

Examples of an antisense nucleic acid capable of specifically suppressing the expression of ABCG1 include: A) a nucleic acid comprising a nucleotide sequence complementary to the nucleotide sequence of an mRNA (mature mRNA or initial transcription product) that encodes ABCG1 or a partial sequence thereof having 12 bases or more in length, (B) a nucleic acid comprising a nucleotide sequence having 12 bases or more in length that is hybridizable specifically with an mRNA (mature mRNA or initial transcription product) that encodes ABCG1 in cells of an animal (preferably human) which is a the subject of treatment, and being capable of inhibiting the translation into the ABCG1 polypeptide in a hybridized state, and the like.

The length of the portion that hybridizes with the target mRNA in the antisense nucleic acid is not particularly limited, as far as the expression of ABCG1 can specifically be suppressed; the length is generally about 12 bases or more, and up to the same length as the full-length sequence of the mRNA (mature mRNA or initial transcription product). Taking into account hybridization specificity, the length is preferably about 15 bases or more, more preferably 18 bases or more. Taking into account the ease of synthesis, antigenicity issues and the like, the length of the portion that hybridizes with the target mRNA is generally about 200 bases or less, preferably about 50 bases or less, more preferably about 30 bases or less. Hence, the length of the portion that hybridizes with the target mRNA is, for example, about 12 to about 200 bases, preferably about 15 to about 50 bases, more preferably about 18 to about 30 bases.

The target nucleotide sequence for the antisense nucleic acid is not particularly limited, as far as the expression of ABCG1 can specifically be repressed or suppressed; the sequence may be the full-length sequence of an mRNA (mature mRNA or initial transcription product) of ABCG1 or a partial sequence thereof (e.g., about 12 bases or more, preferably about 15 bases or more, more preferably about 18 bases or more), or an intron portion of the initial transcription product; however, preferably, the target sequence is located between the 5′-terminal of the mRNA of ABCG1 and the C-terminal of the coding region.

The nucleotide sequence of the portion that hybridizes with the target mRNA in the antisense nucleic acid varies depending on the base composition of the target sequence, and has an identity of generally about 90% or more (preferably 95% or more, most preferably 100%) to the complementary sequence for the target sequence so as to be capable of hybridizing with the mRNA of ABCG1 under physiological conditions.

The size of the antisense nucleic acid is generally about 12 bases or more, preferably about 15 bases or more, more 25 preferably about 18 bases or more. In view of the ease of synthesis, antigenicity issues and the like, the size is generally about 200 bases or less, preferably about 50 bases or less, more preferably about 30 bases or less.

Furthermore, the antisense nucleic acid may be one not only capable of hybridizing with the mRNA or initial transcription product of ABCG1 to inhibit the translation, but also capable of binding to the ABCG1 gene, which is a double-stranded DNA, to form a triplex and inhibit the transcription into mRNA.

Because natural nucleic acids have the phosphodiester bond thereof decomposed readily by nucleases being present in the cells, the siRNA and antisense nucleic acid used in the present invention can also be synthesized using a modified nucleotide such as the thiophosphate form (phosphate bond P═O replaced with P═S) or the 2′-O-methyl form, which are stable to nucleases. Other factors important for the design of the siRNA or antisense nucleic acid include increasing the water solubility and cell membrane permeability and the like; these can also be achieved by improving dosage forms, such as the use of liposomes or microspheres.

An siRNA and antisense nucleic acid capable of specifically suppressing the expression of ABCG1 can be prepared by determining the target sequence on the basis of an mRNA sequence (e.g., nucleotide sequence shown by SEQ ID NO:1) or chromosomal DNA sequence of ABCG1, and synthesizing a nucleotide sequence complementary thereto using a commercially available automated DNA/RNA synthesizer (Applied Biosystems, Beckman and the like). The siRNA can be prepared by separately synthesizing a sense strand and an antisense strand using an automated DNA/RNA synthesizer, and denaturing the strands in an appropriate annealing buffer solution at about 90° C. to about 95° C. for about 1 minute, and then performing annealing at about 30° C. to 70° C. for about 1 to about 8 hours. A longer double-stranded polynucleotide can be prepared by synthesizing complementary oligonucleotide strands in a way such that they overlap with each other, annealing the strands, and then performing ligation with a ligase.

### Vectors:

In a preferred embodiment, the inhibitor of ABCG1 is a nucleic acid carried by an expression vector. In the expression vector, the above-described siRNA or antisense nucleic acid or a nucleic acid (preferably DNA) that encodes the same has been operably linked to a promoter capable of exhibiting promoter activity in cells of a mammal (preferably human).

Any promoter capable of functioning in the cells of the mammal which is the subject of administration can be used. Useful promoters include pol I promoters, pol II promoters, pol III promoters and the like. Specifically, viral promoters such as the SV40-derived initial promoter and cytomegalovirus LTR, mammalian constitutive protein gene promoters such as the .beta.-actin gene promoter, RNA promoters such as the tRNA promoter, and the like are used.

When the expression of an siRNA is intended, it is preferable that a pol III promoter be used as the promoter. Examples of the pol III promoter include the U6 promoter, H1 promoter, tRNA promoter and the like.

At least three methods to generate RNAi-mediated gene silencing in vivo are known and usable in the context of the present invention (Dykxhoorn et al., 2003 for review):

siRNAs with a single sequence specificity can be expressed in vivo from plasmidic or viral vectors using:
Tandem polymerase III promoter that expresses individual sense and antisense strands of the siRNAs that associate in trans;
a single polymerase III promoter that expresses short hairpin RNAs (shRNAs)
a single polymerase II promoter that expresses an imperfect duplex hairpin RNA (pre-miRNA) which is processed by DICER giving rise to a mature miRNA.

The expression vector preferably contains a transcription termination signal, i.e., a terminator region, downstream of the above-described polynucleotide or nucleic acid that encodes the same. Furthermore, a selection marker gene for selection of transformed cells (e.g., genes that confer resistance to drugs such as tetracycline, ampicillin, and kanamycin, genes that compensate for auxotrophic mutations, and the like) can further be included.

Although there is no limitation on the choice of expression vector useful in the present invention, suitable vectors for administration to mammals such as humans include viral vectors such as retrovirus, lentivirus, adenovirus, and adeno-associated virus. Adenovirus, in particular, has advantages such as very high gene transfer efficiency and transferability to non-dividing cells. Because the integration of transgenes into host chromosome is extremely rare, however, the gene expression is transient and generally persists only for about 4 weeks. Considering the persistence of therapeutic effect, it is also preferable to use adeno-associated virus, which offers a relatively high efficiency of gene transfer, which can be transferred to non-dividing cells as well, and which can be integrated into chromosomes via an inverted terminal repeat (ITR).

In a preferred embodiment, the interferent RNA is preferably a shRNA carried by a lentiviral vector that generates lentiviral transduction particles in packaging cell lines.

Alternatively, non-viral vector system may be used and include various formulations such as liposomes, cationic polymers, micelles, emulsions, nanoparticles, and the like. Nanoparticles are described in greater details below. The nucleic acid delivery system can significantly enhance delivery efficiency of the desired nucleic acid into the recipient cells.

### Formulations and Routes of Administration:

The inhibitor of ABCG1 can be formulated within a pharmaceutical composition, in combination with a pharmaceutically acceptable carrier.

Examples of the pharmaceutically acceptable carrier include, but are not limited to, excipients such as sucrose, starch, mannitol, sorbitol, lactose, glucose, cellulose, talc, calcium phosphate, and calcium carbonate; binders such as cellulose, methylcellulose, hydroxypropylcellulose, polypropylpyrrolidone, gelatin, gum arabic, polyethylene glycol, sucrose, and starch; disintegrants such as starch, carboxymethylcellulose, hydroxypropylstarch, sodium-glycol-starch, sodium hydrogen carbonate, calcium phosphate, and calcium citrate; lubricants such as magnesium stearate, Aerosil, talc, and sodium lauryl sulfate; flavoring agents such as citric acid, menthol, glycyrrhizin ammonium salt, glycine, and orange powder; preservatives such as sodium benzoate, sodium hydrogen sulfite, methylparaben, and propylparaben; stabilizers such as citric acid, sodium citrate, and acetic acid; suspending agents such as methylcellulose, polyvinylpyrrolidone, and aluminum stearate; dispersing agents such as surfactants; diluents such as water, physiological saline, and orange juice; base waxes such as cacao butter, polyethylene glycol, and white kerosene; and the like.

When the substance that suppresses the expression or function of ABCG1 is an siRNA or antisense nucleic acid capable of specifically suppressing the expression of ABCG1, or an expression vector capable of expressing said polynucleotide, the pharmaceutical composition may further contain a reagent for nucleic acid transfer in order to promote the transfer of the nucleic acid into a cell. Useful nucleic acid transfer reagents include cationic lipids such as lipofectin, lipofectamine, lipofectamine RNAiMAX, invivofectamine, DOGS (transfectam), DOPE, DOTAP, DDAB, DHDEAB, HDEAB, polybrene, and poly(ethylenimine) (PEI). When a retrovirus is used as the expression vector, retronectin, fibronectin, polybrene and the like can be used as transfer reagents.

Physical techniques can also enhance siRNA uptake at a specific tissue site using electroporation, pressure, mechanical massage, etc. Terminal modification of siRNAs can enhance their resistance to degradation by exonucleases in serum and tissue. Moreover, modification with a suitable ligand can achieve targeted delivery. Several types of carrier for drug delivery have been developed for siRNA in addition to traditional cationic liposome and cationic polymer systems. Ultrasound and microbubbles or liposomal bubbles have also been used in combination with a carrier for siRNA delivery. New materials with unique characteristics such as carbon nanotubes, gold nanoparticles, and gold nanorods have attracted attention as innovative carriers for siRNA. For a recent review, see Higuchi et al, 2010.

In a particular embodiment, the inhibitor, preferably a nucleic acid, is formulated in a nanoparticle. siRNA especially may be delivered by means of nanoparticles. Generally speaking, nanoparticle-based delivery systems are delivery reagents that compact siRNA into particles in the optimal size range of hundreds of nanometers that are on the order of 100,000,000 Daltons in mass. The predominant packaging strategy is to utilize the anionic charge of the siRNA backbone as a scaffold for electrostatic interaction with the delivery reagent. Cationic lipids, cationic polymers, and cationic peptides, which can advantageously be combined with cholesterol, are used to engage the negatively charged phosphodiester backbone and organize large numbers of siRNA molecules into nanoparticle structures prior to cellular treatment in vitro or systemic administration in vivo (Whitehead et al., 2009. See also e.g. WO 2010/080724; US 2006/0240554 and US 2008/0020058).

Beyond cationic motifs required for siRNA nanoparticle formation, additional motifs are applied to the delivery reagent. A large variety of lipids, cell targeting ligands, antibodies, and cell penetrating peptides, to list a few, can be covalently tethered to the cationic packaging motifs so that the resulting nanoparticles that are formed will have cellular delivery properties (Whitehead et al., 2009).

The content of the inhibitor of ABCG1 in the pharmaceutical composition is chosen as appropriate over a wide range without limitations; for example, the content is about 0.01 to 100% by weight of the entire pharmaceutical composition.

Although the dosage of the inhibitor of ABCG1 varies depending on the choice or activity of the active ingredient, dosing route, seriousness of illness, the recipient's drug tolerance, body weight, age, and the like, and cannot be generalized, the dosage is generally about 0.001 mg to about 2.0 g, based on the active ingredient, per day for an adult.

Any route of administration is encompassed. In a particular embodiment, the inhibitor may be in a dosage form adapted for subcutaneous, intradermal, or intramuscular injection.

Injection at a site of excess fat is particularly advantageous. In particular, abdominal injection is preferred, especially when the patient is affected with abdominal obesity. A preferred protocol includes at least one injection in the abdomen at least once a week, or every two days, or every day. Such treatment may be recommended for at least two weeks, preferably at least three weeks, still preferably about a month. The treatment may be extended during several months, e.g. during 2 to 6 months, if needed.

### Diagnostic Methods

The invention further provides a method for determining the level of risk for a subject or patient to develop obesity, especially morbid or abdominal obesity.

Such diagnosis method makes use of a sample from the subject. The sample may be any biological sample derived from a subject, which contains nucleic acids. Examples of such samples include fluids, tissues, cell samples, organs, biopsies, etc. Most preferred samples are blood, plasma, saliva, jugal cells, urine, seminal fluid, etc. The sample may be collected according to conventional techniques and used directly for diagnosis or stored. The sample may be treated prior to performing the method, in order to render or improve availability of nucleic acids or polypeptides for testing. Treatments include, for instant, lysis (e.g., mechanical, physical, chemical, etc.), centrifugation, etc. Also, the nucleic acids may be pre-purified or enriched by conventional techniques, and/or reduced in complexity. Nucleic acids may also be treated with enzymes or other chemical or physical treatments to produce fragments thereof. Considering the high sensitivity of the claimed methods, very few amounts of sample are sufficient to perform the assay.

The sample is preferably contacted with reagents such as probes, or primers in order to assess the presence of an altered gene locus. Contacting may be performed in any suitable device, such as a plate, tube, well, glass, etc. In specific embodiments, the contacting is performed on a substrate coated with the reagent, such as a nucleic acid array. The substrate may be a solid or semi-solid substrate such as any support comprising glass, plastic, nylon, paper, metal, polymers and the like. The substrate may be of various forms and sizes, such as a slide, a membrane, a bead, a column, a gel, etc. The contacting may be made under any condition suitable for a complex to be formed between the reagent and the nucleic acids of the sample.

Any alteration in the ABCG1 gene locus may be searched, especially any form of mutation(s), deletion(s), rearrangement(s) and/or insertions in the coding and/or non-coding region of the locus, especially in the regulatory sequences, like the promoter, alone or in various combination(s). Alterations more specifically include point mutations or single nucleotide polymorphisms (SNP). Deletions may encompass any region of two or more residues in a coding or non-coding portion of the gene locus, such as from two residues up to the entire gene or locus. Typical deletions affect smaller regions, such as domains (introns) or repeated sequences or fragments of less than about 50 consecutive base pairs, although larger deletions may occur as well. Insertions may encompass the addition of one or several residues in a coding or non-coding portion of the gene locus. Insertions may typically comprise an addition of between 1 and 50 base pairs in the gene locus. Rearrangement includes inversion of sequences. The gene locus alteration may result in the creation of stop codons, frameshift mutations, amino acid substitutions, particular RNA splicing or processing, product instability, truncated polypeptide production, etc. The alteration may result in the production of a polypeptide with altered function, stability, targeting or structure. The alteration may also cause a reduction in protein expression or, alternatively, an increase in said production.

In a preferred embodiment, the method of the invention comprises detecting the presence of a nucleotide substitution in the promoter of ABCG1 gene, which may affect the expression of the ABCG1 protein.

In a still preferred embodiment, the nucleotide substitution is at position −134 or −204 from the starting codon of the ABCG1 gene (respectively designated as single nucleotide polymorphism rs1378577 or ss44262232 identified in SEQ ID NO:11 and rs1893590 identified in SEQ ID NO:12), wherein the presence of a T at position −134 and/or a A at position −204 is indicative of a risk of developing obesity. Preferably the presence of haplotype AT for SNPs −204/−134 respectively, is indicative of a higher risk of developing obesity.

The presence of an alteration in the ABCG1 gene locus may be detected by sequencing, selective hybridisation and/or selective amplification.

Sequencing can be carried out using techniques well known in the art, using automatic sequencers. The sequencing may be performed on the complete genes or, more preferably, on specific domains thereof, typically those known or suspected to carry deleterious mutations or other alterations.

Amplification is based on the formation of specific hybrids between complementary nucleic acid sequences that serve to initiate nucleic acid reproduction.

Amplification may be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Preferred techniques use allele-specific PCR or PCR-SSCP. Amplification usually requires the use of specific nucleic acid primers, to initiate the reaction.

Hybridization detection methods are based on the formation of specific hybrids between complementary nucleic acid sequences that serve to detect nucleic acid sequence alteration(s).

A particular detection technique involves the use of a nucleic acid probe specific for wild type or altered gene, followed by the detection of the presence of a hybrid. The probe may be in suspension or immobilized on a substrate or support (as in nucleic acid array or chips technologies). The probe is typically labelled to facilitate detection of hybrids.

In a most preferred embodiment, an alteration in the gene locus is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the alteration of the genes, a sample from a test subject is labelled and contacted with the microarray in hybridization conditions, leading to the formation of complexes between target nucleic acids that are complementary to probe sequences attached to the microarray surface. The presence of labelled hybridized complexes is then detected.

The experimental section below illustrates the invention without limiting its scope:

### Experimental Procedures

### Study Populations

Regression Growth Evaluation Statin Study (REGRESS). The design of the REGRESS trial has been previously described (Jukema et al., 1995). The REGRESS study population is constituted of 886 Caucasian males less than 70 years old, with a minimum 50% obstruction of a major coronary artery, plasma total cholesterol levels between 4 and 8 mmol/L (1.55 and 3.10 g/L) and plasma triglyceride concentrations of less than 4 mmol/L (3.5 g/L).

Obese subjects. Middle-aged (45.71±0.38 years) severely obese patients (n=868; BMI=46.80±0.3 Kg/m2) of Caucasian origin (Sex ratio M/F=0.32) were recruited at the Department of Nutrition at the Pitié-Salpêtrière hospital, Paris, France (Spielmann et al., 2008). All subjects gave their informed written consent to participate in the genetic study, which was approved by the local ethic committee.

### Genotyping

The promoter region of the human ABCG1 gene (NM_{—}207627.1) containing two SNPs at positions −204A/C (ID: rs1893590) and −134T/G (ID: rs1378577) (lida et al., 2002) was amplified by Polymerase Chain Reaction (PCR) using the following forward and reverse primers: 5′-GCTTCACCAGCTCACTTTCC-3′ (SEQ ID NO: 13) and 5′-CATGATGCAATTCCATGTGTA-3′ (SEQ ID NO:14), respectively. Genotype determination was performed as previously described ((Frisdal et al., 2005).

### Animals

ABCG1+/− mice, obtained from Deltagen Inc, San Carlos, Calif., and back-crossed on a C57BI/6 background for 7 generations, were cross-bred to generate ABCG1+/+ and ABCG1−/− mice. Genotyping for ABCG1 was performed according to the protocol from Deltagen. Mice were maintained on sterilized regular chow containing 4.3% (w/w) fat and no cholesterol (RM3, Special Diet Services). To analyse plasma LPL activity, blood was drawn after an overnight fast both before and after an intravenous bolus injection of heparin (100 U/kg).

Animal experiments were performed at the Gorlaeus Laboratories of the Leiden/Amsterdam Center for Drug Research in accordance with the National Laws. All experimental protocols were approved by the Ethics Committee for Animal Experiments of Leiden University.

### Cell Culture

Human Macrophages. Human THP-1 monocytic cells (ATCC) and a THP-1 clone stably transfected with an shRNA targeting human ABCG1, in which ABCG1 expression is stably knocked down (ABCG1 SKD), were cultured and differentiated into macrophage-like cells as previously described (Larrede et al., 2009). Circulating human monocytes were isolated from the blood of individual healthy normolipidemic donors (Etablissement Français du Sang, EFS) on Ficoll gradients (Ficoll-Paque PLUS, GE Healthcare) and subsequently differentiated into human macrophages (HMDM) following the procedure previously reported (Larrede et al., 2009).

3T3-L1 adipocytes. The 3T3-L1 preadipocytes (ATCC) were maintained in Dulbecco's modified Eagle medium (DMEM) supplemented with 10% calf serum and 2 mM glutamine. Differentiation of confluent preadipocytes was initiated with 250 nM insulin, 1250 nM dexamethasone and 250 μM 3-isobutyl-methyl-1-xanthine) in DMEM (4.5 g/L glucose) supplemented with 10% FBS. After 3 days, the culture medium was switched to DMEM (4.5 g/L glucose) supplemented with 10% FBS and 100 nM Insulin for 2 days. Then, 3T3-adipocytes were allowed to mature in DMEM (4.5 g/L glucose) containing 10% FBS, which was replaced every other day for 15 days.

### LPL Activity Assay

Cell culture medium from either macrophage or adipocyte cultures was replaced by a serum-free medium containing 10 U/mL heparin (Choay) and the cells were subsequently incubated for 24 h at 37° C. Culture medium was then collected and stored at −80° C. until determination of LPL activity and cells were lysed overnight in 0.2 N NaOH. Cell protein was quantified using the BCA assay (Pierce). LPL activity was determined using a 50-μl aliquot of culture medium ( 1/10 of total volume), or plasma when indicated, according to the procedure previously described (Stengel et al., 1998). Results are expressed as units of LPL activity (1 U of LPL activity correspond to 1 nmol free fatty acid liberated/min/mg cell protein).

### Flow Cytometry Analysis

Human THP-1 macrophages were cultured in 12-well plates (2.106 cells/well) and incubated in serum-free media in the presence or in the absence of 10 U/mL heparin (Choay) for 24 h at 37° C. following siRNA transfection. Cells were then washed and harvested in cold PBS. After brief centrifugation (2000 rpm, 7 nm) at 4° C., cells were pre-incubated with 100 μl of human Fc Blocker (BD Pharmingen; 1:400 in PBS/BSA 1%) for 10 min at 4° C., and incubated with a monoclonal mouse antibody directed against human LPL (abcam; 1:100) for a further 15 min at 4° C. Cells were washed in 0.1% PBS/BSA and were incubated with a rabbit biotinylated secondary antibody directed against mouse IgG (BD Pharmingen; 1:100 in PBS/BSA 1%) for 15 min. Cells were subsequently incubated with streptavidin-PC7 (Beckman Coulter, 1:20) for 15 min at 4° C. and washed before fixation with PBS/paraformaldehyde (50/50). Prior to flow cytometry analysis, 5 μl of 7-Aminoactinomycin D (7-AAD) (Beckman Coulter) was added to the cell suspension to measure cellular viability. Cells were analyzed on an FC 500 flow cytometer (Beckman Coulter) using Epics XL32 software.

### Immunohistochemistry

After a 24 h-incubation with or without 10 U/mL Heparin (Choay) in serum-free media, control HMDM and ABCG1 KD HMDM were washed in PBS and fixed with 10% phosphate-buffered formalin for 30 minutes. Cells were blocked for 60 minutes with 3% BSA in PBS and then incubated with an anti-hLPL antibody (Abcam; 1:300) overnight at 4° C. After washing, a biotinylated goat anti-mouse IgG secondary antibody (1:1000; BD Pharmingen) was added, followed by the addition of streptavidin-horseradish peroxidase. The signal was enhanced using the tyramide signal amplification (TSA) kit (PerkinElmer) according to the manufacturer's protocol; cells were counterstained for nuclei with DAPI (Invitrogen). Confocal microscopy was performed using a Right confocal microscope Olympus FV-1000 with a 60× objective.

### Cellular Lipid Analysis

Control and ABCG1 KD cells were incubated in the presence or in the absence of 50 μg/ml human VLDL-Prot (d<1.006 g/mL) isolated from normolipidemic plasma by preparative ultracentrifugation (Chapman et al., 1981) and treated with or without 10 μM LPL inhibitor, Tetrahydrolipstatin (THL), (Sigma) for 24 h at 37° C. Quantification of total cellular triglyceride and cholesterol mass was performed as previously described (Milosavljevic et al., 2003).

RNA Interference (RNAi)-Mediated ABCG1 Silencing Using Small Interference (si)RNA

Silencing of ABCG1 expression was performed by application of siRNA oligonucleotides (Dharmacon) targeted to the cDNA sequence of either the human ABCG1 gene (Genebank #AY048757):

Forward
(SEQ ID NO: 3)
5′-UCAUUGGCCUGCUGUACUU-UU-3
Reverse
(SEQ ID NO: 4)
5′-P-AAGUACAGCAGGCCAAUGA-UU-3′
or mouse Abcg1 gene (Genebank #NM009593):
Forward
(SEQ ID NO: 15)
5′-GCGAAGCUGUACCUGGAUU-UU
Reverse
(SEQ ID NO: 16)
5′-P-AAUCCAGGUACAGCUUCGC-UU-3′

Ten-day differentiated HMDM were grown in 24-well plates and transfected with 50 nM control siRNA (Dharmacon) or siRNA targeting human ABCG1 using lipofectamine RNAiMax (Invitrogen) according to the manufacturer's instructions. Transfection of 3T3-L1 preadipocytes and mature adipocytes with siRNA was achieved using the Nucleofector® technology (Lonza) according to the manufacturer's protocol. For each experiment, 2×106 cells and 100 pmol siRNA were diluted in 100 μl of V solution and processed with A-033 program.

### RNA Extraction and Gene Expression Analysis

Twenty-four hours following transfection with siRNA, Control and ABCG1 KD cells were washed twice with cold PBS and total RNA was extracted using a NucleoSpin RNA II kit (Macherey-Nagel) according to the manufacturer's instructions. Reverse transcription of RNA and real time quantitative PCR using a LightCycler LC480 (Roche) were performed as previously described (Larrede et al., 2009). Expression data were based on the crossing points calculated with the software for LightCycler data analysis and corrected for PCR efficiencies of the target and the reference gene. When indicated, data were expressed as a fold change in mRNA expression relative to control values.

### Western Blot Analysis

Cell proteins were extracted using 200 μL M-PER reagent (Pierce) containing protease inhibitors and were subsequently separated on a 4-12% Bis-Tris gel (Invitrogen). Proteins (25 μg per lane) were transferred to nitrocellulose and the membrane was blocked with Casein blocker solution for 1 h. ABCG1 was detected using rabbit anti-hABCG1 (NB400-132; Novus) at 1:500 and goat anti-rabbit/HRP (Dako) at 1:15000.

### DNA Constructs

A 1056-bp fragment corresponding to the region from +51 to −1005 of the human ABCG1 gene was amplified by PCR from individuals homozygous for either the −134T or −134G and either the −204A or −204C allele using the following upstream and downstream primers, 5′-CGTGCATGAATCACAAAAA-3′ (SEQ ID NO:17) and 5′-CACCACTGCAGGCATGTAA-3′ (SEQ ID NO:18), respectively. The PCR product was purified and subcloned using the TA overhang into the pCR2.1 vector (Invitrogen). Then, a 1138-bp SacI-XhoI fragment containing the human ABCG1 promoter and a portion of the pCR2.1 polylinker was isolated and cloned into the SacI-XhoI cut pGL3-Basic vector (Promega), generating the phABCG1-AT and phABCG1-GC constructs. The orientation and the integrity of the inserts were verified by sequencing.

The functionality of both SNPs was tested by the transient transfection of either the phABCG1-AT construct or the phABCG1-GC construct together with a β-galactosidase expression vector (pCMV.Sport-β gal, Invitrogen) in HepG2 cells as previously described (Le Goff et al., 2002).

### Statistical Analyses

Allele frequencies were calculated from the genotype counts. In the REGRESS cohort, the observed genotype counts were compared with those expected under Hardy-Weinberg equilibrium with a χ²-test with one degree of freedom. A single or dual ABCG1 polymorphism genotype effect was tested by analysis of variance and a backward regression analysis, respectively. Haplotype effects were estimated using a method described by Tanck et al. (Souverein et al., 2005). In the obese subject cohort, linkage disequilibrium between both SNPs was calculated with Haploview 4.1; haplotypes were reconstructed with Famhap 18. Associations between phenotypes and genotypes or haplotypes were tested with multivariate linear regression models. All models were adjusted for age and sex. All phenotypes were transformed to log 10 before testing for associations. Association tests were performed with R 2.8.2. Throughout, p-values <0.05 were interpreted as significant.

### Example 1

### ABCG1 Genotype is Associated with Plasma Lipoprotein Lipase Activity in Regress

The inventors have analyzed the distribution of ABCG1 polymorphisms in the dyslipidemic population of the lipid-lowering Regression Growth Evaluation Statin Study (REGRESS) study population.

Analysis of the potential association of the −134T/G and −204A/C variants with plasma lipid levels and angiographic parameters revealed that neither of the two ABCG1 SNPs were associated with BMI, plasma Cholesteryl Ester Transfer Protein (CETP) concentration, plasma lipid levels (total cholesterol, LDL-C, HDL-C, triglycerides and Lp(a)) nor with angiographic parameters (Minimum Segment Diameter and Mean Obstruction Diameter) (Table 1).

TABLE 1
Plasma and angiographic parameters as a function of
the -204A/C and -134T/G ABCG1 polymorphisms in REGRESS.

| | ABCG1 -204A/C | | | | | | |
|---|---|---|---|---|---|---|---|
| | AA | | AC | | CC | | |
| | n | Mean (±SD) | n | Mean (±SD) | n | Mean (±SD) | P |
| Lipid parameters | | | | | | | |
| BMI (Kg/m²) | 314 | 25.8 (±2.75) | 225 | 26.3 (±2.52) | 30 | 25.88 (±2.32) | 0.796 |
| Total Cholesterol (mmol/l) | 327 | 6.03 (±0.9) | 241 | 6.01 (±0.89) | 33 | 6.02 (±0.81) | 0.982 |
| LDL-C (mmol/l) | 325 | 4.29 (±0.81) | 237 | 4.27 (±0.81) | 33 | 4.27 (±0.7) | 0.964 |
| HDL-C (mmol/l) | 326 | 0.93 (±0.23) | 237 | 0.91 (±0.21) | 33 | 0.98 (±0.23) | 0.141 |
| Triglycerides (In[mmol/l]) | 326 | 0.49 (±0.44) | 241 | 0.51 (±0.44) | 33 | 0.45 (±0.42) | 0.510 |
| CETP (μg/ml) | 252 | 1.93 (±0.5) | 185 | 1.93 (±0.6) | 21 | 1.85 (±0.47) | 0.508 |
| Lp(a) | 272 | 5.41 (±1.34) | 205 | 5.35 (±1.26) | 28 | 5.61 (±21.3) | 0.362 |
| LPL activity (mU/ml) | 264 | 108.77 (±42.53) | 195 | 108.56 (±41.84) | 29 | 134.31 (±53.58)* | 0.002* |
| LPL mass (In[μg/ml]) | 252 | 5.72 (±1.12) | 186 | 5.82 (±1.09) | 21 | 5.48 (±0.88) | 0.253 |
| Angiography | | | | | | | |
| MSD (mm) | 231 | 2.81 (±0.44) | 187 | 2.82 (±0.5) | 28 | 2.85 (±0.52) | 0.645 |
| MOD (mm) | 235 | 1.88 (±0.56) | 190 | 1.89 (±0.53) | 28 | 1.94 (±0.46) | 0.639 |
| | ABCG1 -134T/G | | | | | | |
| | TT | | TG | | GG | | |
| | n | Mean (±SD) | n | Mean (±SD) | n | Mean (±SD) | P |
| Lipid parameters | | | | | | | |
| BMI (Kg/m²) | 367 | 25.89 (±2.74) | 182 | 26.16 (±2.55) | 22 | 26.44 (±1.82) | 0.422 |
| Total Cholesterol (mmol/l) | 385 | 6.03 (±0.92) | 196 | 6.01 (±0.82) | 22 | 6.06 (±0.91) | 0.841 |
| LDL-C (mmol/l) | 383 | 4.29 (±0.82) | 192 | 4.27 (±0.75) | 22 | 4.29 (±0.73) | 0.964 |
| HDL-C (mmol/l) | 384 | 0.94 (±0.23) | 192 | 0.9 (±0.21) | 22 | 0.96 (±0.25) | 0.841 |
| Triglycerides (In[mmol/l]) | 384 | 0.48 (±0.44) | 196 | 0.54 (±0.43) | 22 | 0.51 (±0.41) | 0.900 |
| CETP (μg/ml) | 291 | 1.92 (±0.49) | 153 | 1.95 (±0.62) | 13 | 1.87 (±0.52) | 0.732 |
| Lp(a) | 322 | 5.35 (±1.32) | 166 | 5.47 (±1.26) | 18 | 5.33 (±1.45) | 0.827 |
| LPL activity (mU/ml) | 307 | 109.67 (±43.17) | 161 | 107.79 (±41.46) | 19 | 137.74 (±55.11)* | 0.005* |
| LPL mass (In[μg/ml]) | 292 | 5.73 (±1.1) | 153 | 5.81 (±1.11) | 13 | 5.5 (±0.95) | 0.404 |
| Angiography | | | | | | | |
| MSD (mm) | 282 | 2.8 (±0.43) | 152 | 2.84 (±0.53) | 16 | 2.77 (±0.49) | 0.725 |
| MOD (mm) | 288 | 1.87 (±0.56) | 153 | 1.92 (±0.55) | 16 | 1.85 (±0.38) | 0.788 |

MSD indicates mean segment diameter;
MOD, mean obstruction diameter.
P values suppose a recessive model for statistical analyses.

Interestingly however, the two SNPs were strongly associated with LPL activity (p<0.005); individual homozygous for the less frequent allele of each polymorphism (−204CC and −134GG) displayed with the highest LPL activity. However, neither the −204CC nor the −134GG genotypes were associated with plasma LPL mass. A multilocus analysis with both ABCG1 SNPs indicated that the −134T/G and −204A/C SNPs were not independent predictors of plasma LPL activity, thereby suggesting that only a single SNP is functional and that the effect of the other is due to linkage disequilibrium (LD).

These findings therefore reveal that ABCG1 SNPs are associated with plasma LPL activity in humans, an effect that appears to be independent of LPL mass.

### Example 2

### Plasma Lipoprotein Lipase Activity is Subnormal in Abcg1 KO Mice

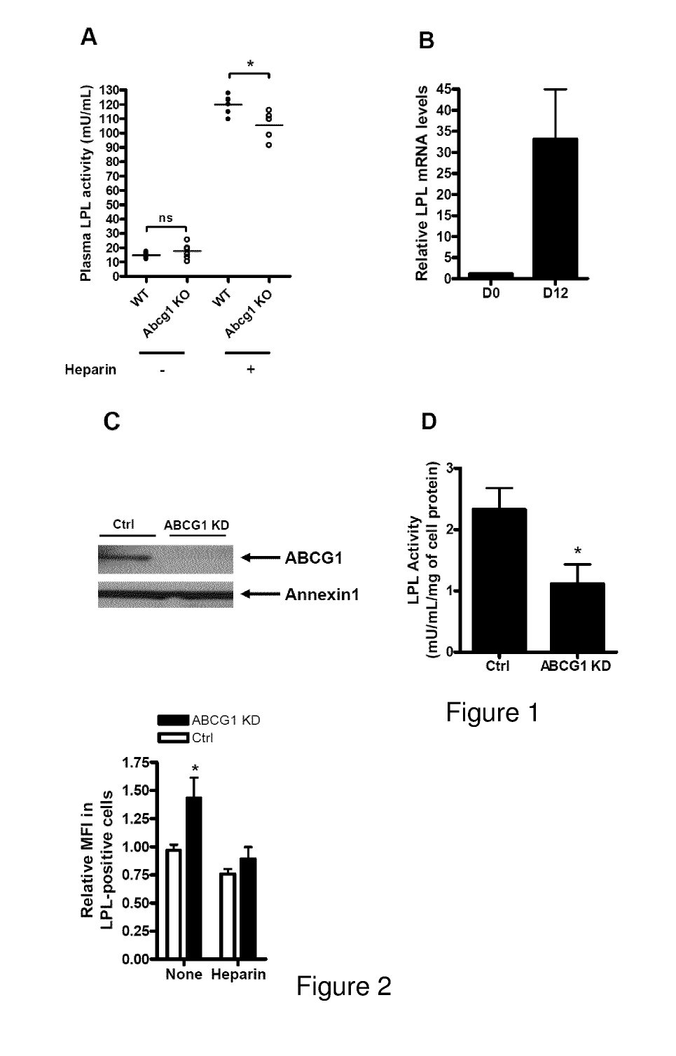

In order to investigate the possible relationship between ABCG1 and LPL activity, the inventors measured plasma LPL activity in ABCG1 deficient mice (Abcg1 KO) before and after heparin injection (FIG. 1A). When fed a chow diet, plasma LPL activity was significantly lower (−12%, p<0.01) in Abcg1 KO mice as compared to WT mice after heparin injection, whereas LPL activity in pre-heparin plasma from WT and Abcg1 KO mice was indistinguishable. Consistent with data from analysis of ABCG1 SNPs in REGRESS, these findings indicate that cellular ABCG1 expression is associated with plasma LPL activity in mice, in which plasma LPL activity was attenuated in the absence of ABCG1.

### Example 3

### Silencing of ABCG1 in Human Macrophages Leads to Reduction in Secreted LPL Activity as a Consequence of Retention at the Cell Surface

The monocyte-derived macrophage is an important cell in the development of atherosclerosis, and macrophages and macrophage-derived foam cells constitute the primary source of LPL within the atherosclerotic lesion (Takahashi et al., 1995). Moreover, the expression of either ABCG1 or LPL in macrophages has been demonstrated to play a significant role in atherogenesis in mice (Babaev et al., 1999; Kennedy et al., 2005; Out et al., 2007; Van Eck et al., 2000; Yvan-Charvet et al., 2007). As shown in FIG. 1B, human monocyte-derived macrophages (HMDM) differentiated for 12 days in the presence of human M-CSF displayed ˜100-fold higher amounts of LPL mRNA than freshly isolated human monocytes. In order to explore the potential relationship between ABCG1 expression and LPL activity in human macrophages, the inventors silenced ABCG1 expression in HMDM using siRNA specific for the human ABCG1 gene; predictably almost complete abolition of ABCG1 expression occurred (FIG. 1C). As shown in FIG. 1D, secreted LPL activity from ABCG1 Knockdown (KD) HMDM was significantly reduced (−52%, p=0.02) as compared to control HMDM after heparin treatment, clearly indicating that ABCG1 expression impacts secreted LPL activity from human macrophages to a major degree.

In order to determine whether the reduction in LPL activity observed in ABCG1 KD HMDM results from decrease in LPL expression, the inventors next quantified LPL mRNA levels by real-time quantitative PCR in ABCG1-deficient macrophages. Interestingly, the knockdown of ABCG1 expression in human macrophages was not accompanied by reduction in LPL expression. Rather a minor increment in LPL expression (+28%, p<0.05) was detected in ABCG1 KD HMDM as compared to control HMDM. Such an effect was also observed in ABCG1 KO BMDM. These data suggest that ABCG1 does not control LPL activity through modulation of LPL mRNA expression in human macrophages.

Since attenuated LPL activity in ABCG1 KD human macrophages did not result from reduction in cellular LPL mRNA expression, the inventors next examined the possibility that secretion of LPL was impaired in macrophages when ABCG1 expression was deficient. Visualization of LPL in HMDM by confocal microscopy revealed that LPL expression at the cell surface of ABCG1 KD HMDM was much more pronounced than in control cells. Treatment with heparin markedly reduced the immunorecognition of LPL at the cell surface; however the abundance of LPL detected at the cell surface of ABCG1 KD HMDM still remained higher than that in control HMDM.

Quantification of LPL at the cell surface by fluorescence-activated cell sorting analysis in human THP-1 macrophages (FIG. 2) revealed that the expression of LPL was markedly increased at the cell surface of ABCG1 KD THP-1 macrophages as compared to control cells (+48%, p<0.05).

Clearly then, invalidation of ABCG1 expression in human macrophages leads to cell surface retention of LPL at heparin-resistant sites.

### Example 4

### ABCG1 Expression is Essential for LPL-Mediated Lipid Accumulation in Human Macrophages

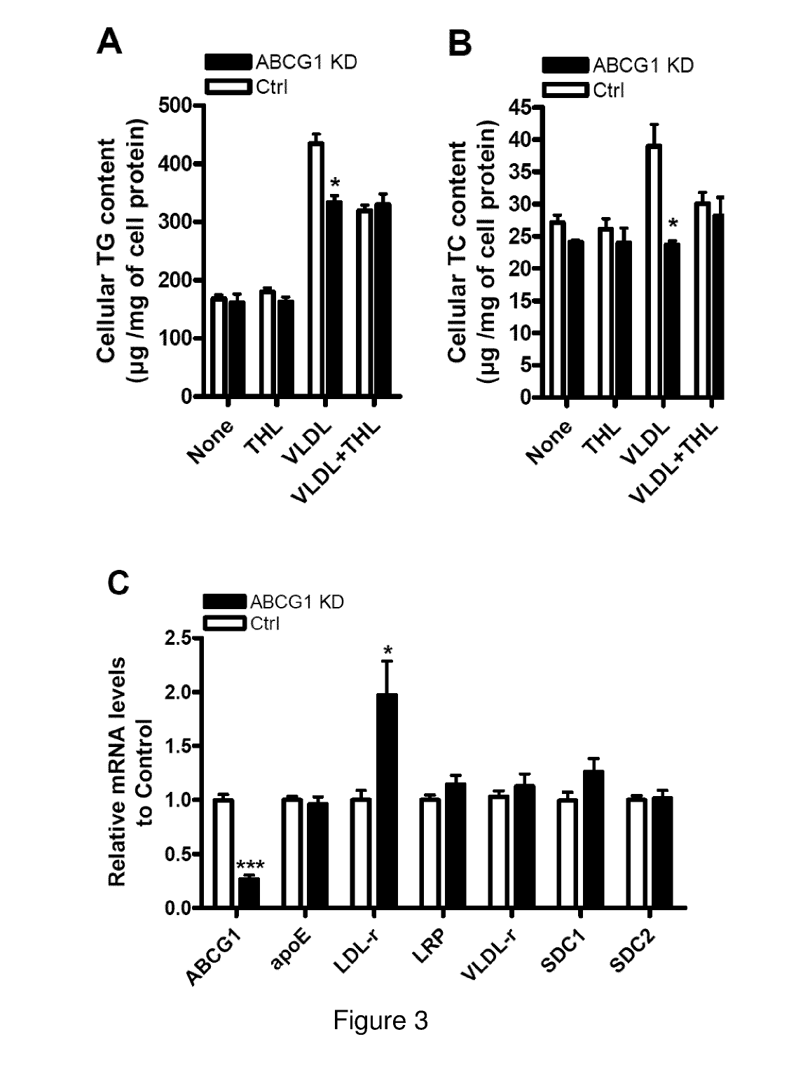

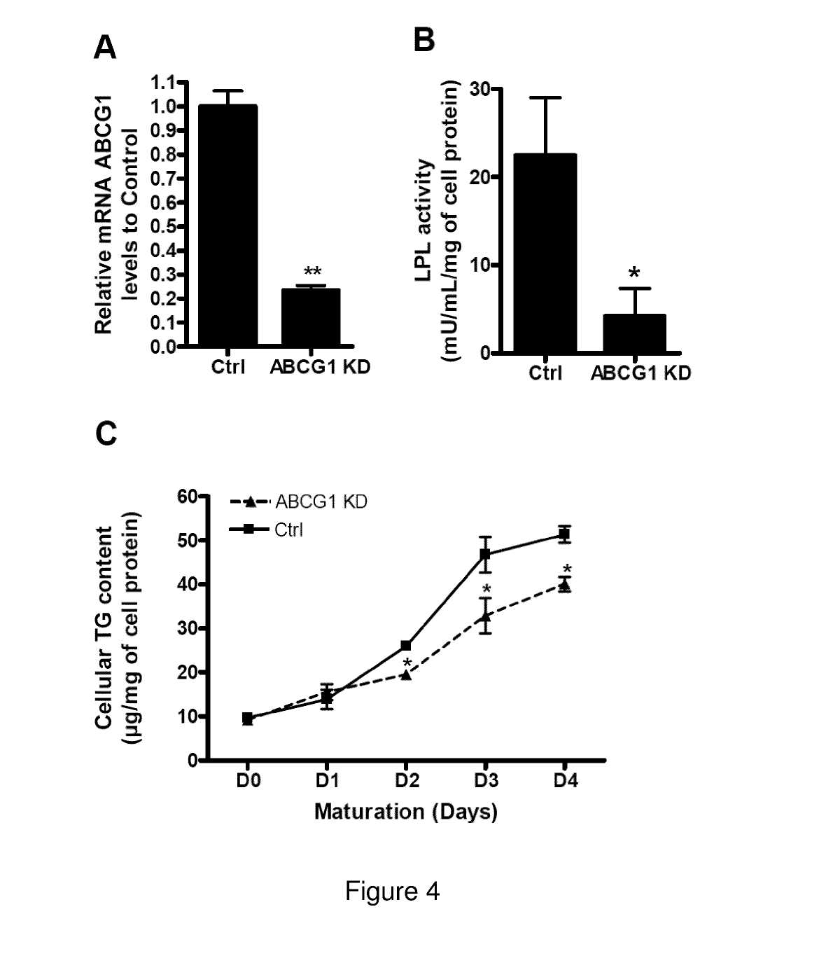

It is established that LPL is a key factor in promoting macrophage foam cell formation, mainly through its role in facilitating cellular lipoprotein uptake (Babaev et al., 1999; Milosavljevic et al., 2003). To evaluate the potential pathophysiological relevance of the interaction between ABCG1 and LPL activity in foam cell formation, the capacity of Very Low Density Lipoprotein (VLDL) to mediate cellular lipid accumulation was evaluated in control and ABCG1 KD HMDM (FIGS. 3A and B). Incubation of primary human macrophages with human VLDL for 24 hours led to marked elevation in cellular triglyceride (FIG. 4A) and total cholesterol (FIG. 3B) contents in control HMDM (+258% and +46% respectively, p<0.001). Specific inhibition of LPL activity by 10 μM tetrahydrolipstatin (THL) reduced both TG and TC accumulation induced by VLDL (−43% and −75%, respectively), thus illustrating the major role of LPL in cellular lipid accumulation. More strikingly, the VLDL-induced accumulation of TG in ABCG1 KD HMDM was markedly reduced (−38%, p<0.01) as compared to control cells whereas that of cholesterol was completely abolished (FIG. 3A); such an effect was not observed when LPL activity was inhibited by THL. Moreover, the capacity of THL to inhibit LPL-mediated lipid uptake was abolished in ABCG1 KD macrophages, thus strengthening the specific concerted interaction between ABCG1 and LPL in VLDL uptake. Such cooperation between ABCG1 and LPL in the uptake of modified LDL (acLDL and oxLDL) was however not observed in human macrophages (data not shown).

It is relevant that the relative mRNA levels coding for core proteins of heparan sulfate proteoglycans (Syndecan1 (SDC1), Syndecan2 (SDC2)), and cellular lipoprotein receptors (VLDL-receptor (VLDL-r), Lipoprotein Related Receptor (LRP)) and apolipoprotein E (apoE), potential partners for LPL in VLDL uptake (Lindqvist et al., 1983) were not altered in ABCG1 KD human macrophages (FIG. 3D). Interestingly, LDL-receptor (LDL-r) expression was significantly induced in ABCG1 KD macrophages (2-fold; p<0.01) as compared to control cells, probably as a result of the activation of the SREBPs in response to a fall in intracellular cholesterol content in these cells.

Taken together, these data clearly indicate that ABCG1 plays a critical role in LPL-dependent lipid accumulation, and especially in that of TG, in human macrophages.

### Example 5

### ABCG1 Promotes Cellular Triglyceride Storage in Adipocytes

To further explore the potential pathophysiological relevance of ABCG1 to cellular TG accumulation mediated by LPL, the inventors next investigated the possibility that ABCG1 might be implicated in TG storage in adipocytes. Indeed, LPL produced by adipocytes was reported to exert a major role in TG accumulation in these cells by hydrolyzing TG from circulating lipoproteins and thus generating fatty acids which drive intracellular TG synthesis and adipocyte maturation (Gonzales and Orlando, 2007).

As expected, siRNA-mediated inhibition of ABCG1 (−77%, p<0.0005) in mature 3T3-L1 adipocytes (FIG. 4A) led to a marked reduction in LPL activity (−81%, p<0.05) in media from ABCG1 KD adipocytes (FIG. 4B) as compared to control cells, thus confirming that ABCG1 directly interacts with LPL activity in fat cells. More strikingly, the silencing of ABCG1 expression in preadipocytes (ABCG1 KD) prior to the addition of the adipocyte differentiation cocktail (Day 0, D0) led to a marked reduction in intracellular TG accumulation during adipocyte maturation as compared to control cells (FIG. 4C; −22%, p<0.05 after 4 days of differentiation).

### Example 6

### ABCG1 Genotype is Associated with BMI in Obese Individuals

The role of ABCG1 in TG storage in adipocytes described herein led the inventors to propose that ABCG1 expression may be related to the development of fat mass, and therefore potentially obesity, in humans. Genotyping of the −134T/G and −204A/C ABCG1 SNPs was therefore performed in a population of 868 middle-aged severely obese patients (BMI=46.80±0.3 Kg/m2). The relative allele frequencies for both ABCG1 SNPs in the population of obese individuals were similar to those observed in the REGRESS cohort (−134T/G (0.78/0.22) and −204A/C (0.73/0.27)). Importantly, the two ABCG1 SNPs were found to be significantly associated with BMI in individuals homozygous for the most frequent allele for each polymorphism (−134TT and −204AA), and who displayed the highest BMI (Table 2).

TABLE 2
Analysis of BMI (kg/m²) as a function of −134T/G and −204A/C ABCG1
polymorphisms in obese patients.

| ABCG1 SNPs | |
|---|---|
| −134 T/G | −204 A/C |

| genotype | G/G & G/T | T/T | A/A | A/C & C/C |
|---|---|---|---|---|
| mean | 45.45 | 47.49 | 47.43 | 45.86 |
| SD | 8.34 | 9.05 | 9.19 | 8.31 |
| n | 321 | 505 | 411 | 340 |
| p | 8 · 10⁻⁴ | | 0.01 | |

The effect of each SNP on BMI was analyzed by linear regression in an additive, dominant and recessive manner. All models were adjusted for age and sex.

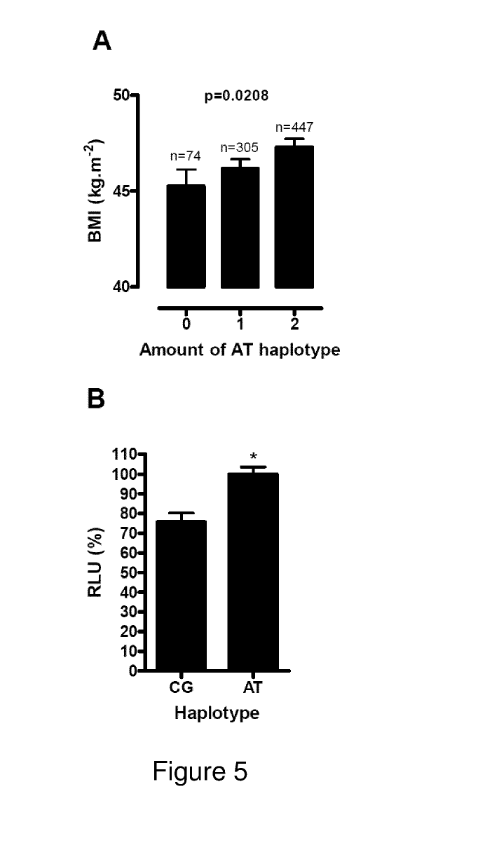

Haplotype analysis confirmed that the AT haplotype (−204A/−134T) was significantly associated with BMI (p=0.0208); moreover, BMI increased in parallel with increase in the amount of the AT haplotypes (FIG. 5A).

In order to validate the overall mechanism, the in vitro functionality of each haplotype was evaluated by transient transfection in HepG2 cells using a reporter gene plasmid driven by the 1056 proximal human ABCG1 promoter region (+11/−1056 bp). The experimental findings indicated that the construct carrying the AT haplotype displayed significantly higher promoter activity (+25%, p<0.0005) than that carrying the CG haplotype (FIG. 5B).

Taken together, these results indicate that the AT haplotype for the −204/−134 ABCG1 SNPs is associated not only with an increased ABCG1 promoter activity, but also with elevated BMI in obese individuals.

### Example 7

### A Higher Expression of ABCG1 in Adipose Tissue is Associated to Increased Features of Obesity in Obese Patients

### 7.1. Materials and Methods.

### RNA Extraction, Reverse-Transcription and Quantitative-PCR.

The adipose tissue pieces were sampled, after an overnight fast, in the s.c. peri-umbilical by needle biopsy under local anesthesia (1% xylocalne). Biopsies were washed and stored in RNA Later preservative solution (Qiagen) at −80° C. until analysis. Total RNA was extracted from adipose tissue biopsies using the RNeasy total RNA minikit (Qiagen). Total RNA concentration and quality was confirmed using the Agilent 2100 bioanalyzer (Agilent Technologies). Then, 500 ng of RNA was reverse transcribed with 75 ng of random hexamer using 200 units of M-MLV reverse transcriptase. An initial denaturation step for 5 min at 68° C. was followed by an elongation phase of 1 h at 42° C.; the reaction was completed by 5-min incubation at 68° C.

Real time quantitative PCR was performed using a LightCycler LC480 (Roche). The reaction contained 2.5 ng of reverse transcribed total RNA, 150 pmol of forward and reverse primers and 5 μl of Master Mix SYBR-Green, in a final volume of 10 μl. Samples underwent the standard PCR protocol. Crossing point (CP) values for genes of interest were normalized to human non-POU domain containing, octamer-binding housekeeping gene (NONO), human α-tubulin (TUBA) and human heat shock protein 90 kDa alpha (cytosolic), class B member 1 (HSP90AB1) or mouse hypoxanthine phosphoribosyltransferase 1 (HPRT 1). Expression data were based on the crossing points calculated with the software for LightCycler data analysis and corrected for PCR efficiencies of the target and the reference gene.

### Adipocyte Diameter Measurements.

Adipose tissue pieces were minced and immediately digested by 200 μg/mL collagenase (Sigma) for 30 min at 37° C. For cell size measurements, adipocyte suspensions were then visualized under a light microscope attached to a camera and computer interface. Adipocyte diameters were measured by using PERFECT IMAGE software (Numeris). Mean diameter was defined as the median value for the distribution of adipocyte diameters of ≧250 cells.

### 7.2. Results.

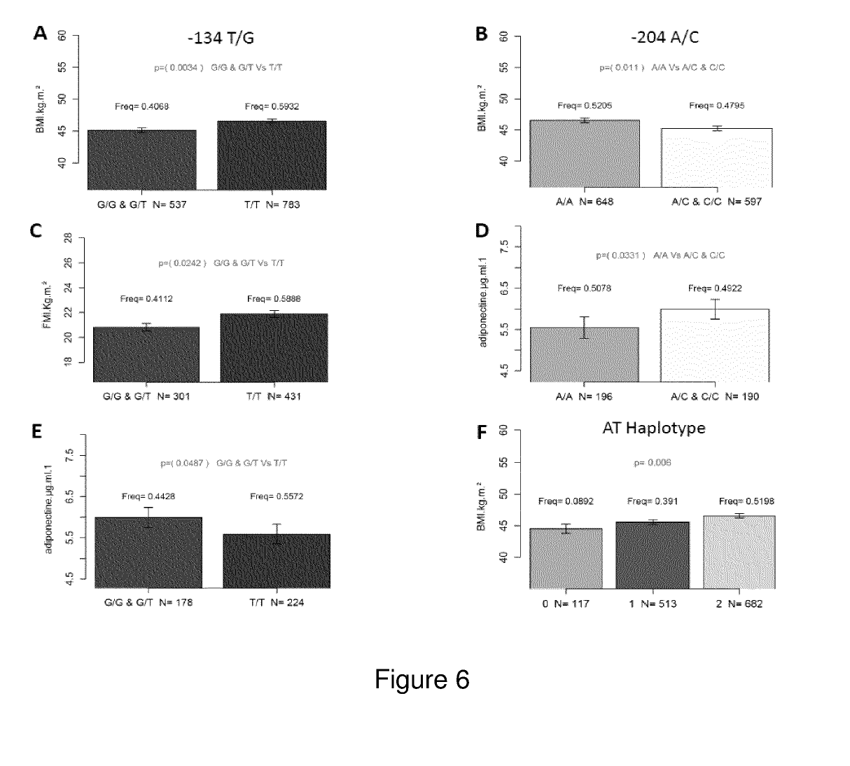

Genotyping of the −134T/G (rs1378577) and −204A/C (rs1893590) ABCG1 SNPs was performed in an additional population of 962 middle-aged severely obese patients (BMI=46.80±0.3 Kg/m2) in order to replicate results shown in FIG. 5A. Analysis of the effect of both SNPs on the total population (this study and the initial application) confirmed that the −134T/G and −204A/C ABCG1 SNPs were significantly associated with individuals homozygous with the most frequent allele for each polymorphism (−134TT and −204AA) displaying the highest BMI. Haplotype (FIGS. 6A and 6B). Haplotype analysis confirmed that the AT haplotype (−204A/−134T) was significantly associated with BMI (p=0.006), with BMI increased in parallel with increase in the amount of the AT haplotype (FIG. 6E). Interestingly, in addition to an increased BMI, obese individuals carrying the −134TT genotype (most frequent allele) also displayed the highest fat mass index (FMI, FIG. 6C) and the lowest plasma adiponectin levels, such an effect being equally observed in subjects carrying the −204AA genotype (most frequent allele) (FIG. 6D).

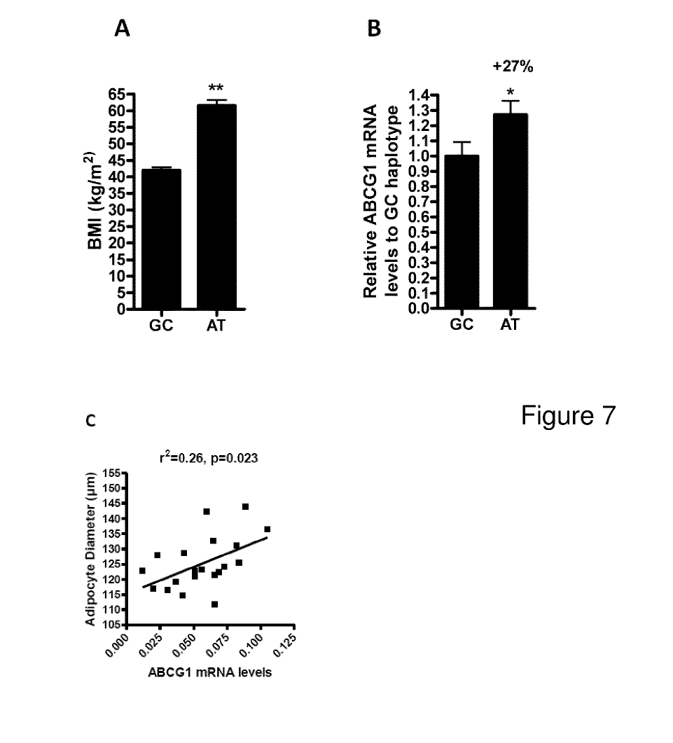

In order to validate the hypothesis that an elevated expression of ABCG1 might be associated to an increased fat mass formation and obesity in obese subjects, ABCG1 expression was analyzed in biopsies of adipose tissues isolated from obese patients displaying either the AT or GC haplotype. Coherent with the analysis of the ABCG1 SNPs in the total population of obese patients, selected individuals carrying the AT haplotype displayed a much higher BMI (+47%, p<0.0001) than those carrying the GC haplotype (FIG. 7A). In addition, mRNA levels of ABCG1 were 27% (p<0.05) more elevated in adipose tissues from those patients (AT haplotype, FIG. 7B), a result in total accordance with the data obtained from the in vitro analysis of ABCG1 promoter activity (+25% AT vs GC, p<0.0005) (FIG. 5B). More strikingly, the inventors observed that ABCG1 expression in adipose tissue from obese patients was positively correlated to the adipocyte diameter (r2=0.26, p=0.023, FIG. 7C), a hallmark of adipocyte hypertrophy in obesity.

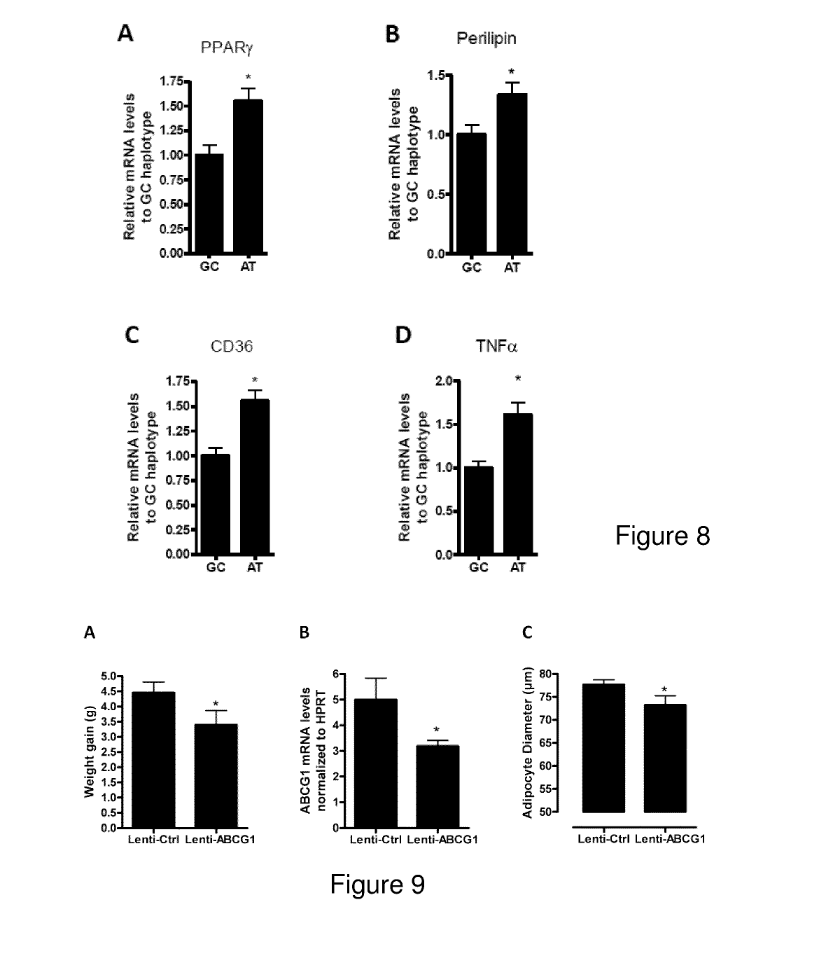

In addition, the inventors observed that amounts of mRNA coding for genes involved in adipocyte differenciation (PPARγ), maturation (CD36, perilipin) and inflammation (TNFα) were equally increased in adipose tissue from obese patients carrying the AT haplotype as compared to those carrying the GC haplotype (FIGS. 8A to 8D).

Taken together, those results indicated that the AT haplotype for the −204/−134 ABCG1 SNPs in obese individuals is associated with a higher expression of ABCG1 and of genes involved in adipocyte differentiation and maturation in adipose tissue concomitant to an increased adipocyte diameter and BMI observed in those patients.

### Example 8

### Local Delivery of siRNA Targeting ABCG1 Expression in Adipose Tissue In Vivo Led to a Rapid Reduction of Gain Weight

### 8.1. Materials and Methods.

Injection of siRNA Targeting ABCG1 Expression in Adipose Tissue In Vivo.

Four-week aged male C57BL/6 mice (Janvier) were fed on a high fat diet (45% fat, Brogaarden Diet#TD12451) for 4 weeks before the day of injection. At the day of injection, mice were weighted and anesthetized with isoflurane and maintained under anesthesia during the surgical procedure. A sub-abdominal incision was operated and epididymal fat pads was injected with 100 μl of lentiviral particles (1.4×10⁵ lentiviral transducing particles per milliliter) encoding either a short-hairpin RNA (shRNA) designed to knock down mouse ABCG1 expression (Santa Cruz) or control shRNA lentiviral particles encoding a shRNA that will not lead to the degradation of any known cellular mRNA (Santa Cruz) using a 30 gauge needle. Then injected epididymal fat pads were replaced in the sub-abdominal cavity and the incision was sutured. Mice were fed for an additional 4-week period on a high fat diet (60% fat, Brogaarden Diet#TD12492) until the day of sacrifice. At the day of sacrifice, mice were weighted, euthanized and epididymal adipose tissue were isolated for RNA extraction and adipocyte diameter measurements.

### 8.2. Results

In order to validate the proof of concept that the RNAi-mediated inhibition of ABCG1 offers a valid and efficient therapeutic approach to achieve weight loss in obese individuals, the inventors tested the impact of the local delivery of siRNA targeting ABCG1 expression in adipose tissue on gain weight in vivo in mice. To achieve this goal, epididymal adipose tissue from C57BL/6 mice fed a high fat diet was injected with lentiviral particles encoding either a short-hairpin RNA (shRNA) designed to knock down mouse ABCG1 expression (lenti-ABCG1) by RNAi or control shRNA lentiviral particles (lenti-Ctrl). As shown in FIG. 9A, gain weight in lenti-ABCG1 mice was reduced by 24% (p<0.05) no longer than 4 weeks after the injection as compared to lenti-Ctrl. Analysis of ABCG1 mRNA levels confirmed that the expression of ABCG1 was reduced by 40% in adipose tissue from lenti-ABCG1 mice (FIG. 9B). More strikingly, the diameter of adipocytes isolated in epididymal adipose tissue from lenti-ABCG1 mice (according to the method of Example 7) was significantly smaller than that from epididymal adipose tissue in lenti-Ctrl mice (FIG. 9C), a result in total agreement with our results obtained in adipose tissue from obese patients (FIG. 7C).

These results show that the local delivery of siRNA inhibiting ABCG1 expression in vivo is a valid pharmacological strategy to reduce gain weight in vivo.

### REFERENCES

1. Babaev, V. R., Fazio, S., Gleaves, L. A., Carter, K. J., Semenkovich, C. F., and Linton, M. F. (1999). Macrophage lipoprotein lipase promotes foam cell formation and atherosclerosis in vivo. J Clin Invest 103, 1697-1705.
2. Baldan, A., Bojanic, D. D., and Edwards, P. A. (2009). The ABCs of sterol transport. J Lipid Res 50 Suppl, S80-85.
3. Buchmann, J., Meyer, C., Neschen, S., Augustin, R., Schmolz, K., Kluge, R., Al-Hasani, H., Jurgens, H., Eulenberg, K., Wehr, R., et al. (2007). Ablation of the cholesterol transporter adenosine triphosphate-binding cassette transporter G1 reduces adipose cell size and protects against diet-induced obesity. Endocrinology 148, 1561-1573.
4. Chapman, M. J., Goldstein, S., Lagrange, D., and Laplaud, P. M. (1981). A density gradient ultracentrifugal procedure for the isolation of the major lipoprotein classes from human serum. J Lipid Res 22, 339-358.
5. Dykxhoorn D. M., Novina C. D., Sharp P. A. (2003) Killing the messenger: short RNAs that silence gene expression. Nat Rev Mol Cell Biol. 4, 457-467.
6. Frisdal, E., Klerkx, A. H., Le Goff, W., Tanck, M. W., Lagarde, J. P., Jukema, J. W., Kastelein, J. J., Chapman, M. J., and Guerin, M. (2005). Functional interaction between −629C/A, −971G/A and −1337C/T polymorphisms in the CETP gene is a major determinant of promoter activity and plasma CETP concentration in the REGRESS Study. Hum Mol Genet 14, 2607-2618.
7. Gonzales, A. M., and Orlando, R. A. (2007). Role of adipocyte-derived lipoprotein lipase in adipocyte hypertrophy. Nutr Metab (Lond) 4, 22.
8. Higuchi Y, Kawakami S, Hashida M., BioDrugs. 2010 June; 24(3):195-205. Strategies for in vivo delivery of siRNAs: recent progress.
9. Hindorff, L. A., Sethupathy, P., Junkins, H. A., Ramos, E. M., Mehta, J. P., Collins, F. S., and Manolio, T. A. (2009). Potential etiologic and functional implications of genome-wide association loci for human diseases and traits. Proc Natl Acad Sci U S A 106, 9362-9367.
10. Iida, A., Saito, S., Sekine, A., Mishima, C., Kitamura, Y., Kondo, K., Harigae, S., Osawa, S., and Nakamura, Y. (2002). Catalog of 605 single-nucleotide polymorphisms (SNPs) among 13 genes encoding human ATP-binding cassette transporters: ABCA4, ABCA7, ABCA8, ABCD1, ABCD3, ABCD4, ABCE1, ABCF1, ABCG1, ABCG2, ABCG4, ABCG5, and ABCG8. J Hum Genet 47, 285-310.
11. Jukema, J. W., Bruschke, A. V., van Boven, A. J., Reiber, J. H., Bal, E. T., Zwinderman, A. H., Jansen, H., Boerma, G. J., van Rappard, F. M., Lie, K. I., and et al. (1995). Effects of lipid lowering by pravastatin on progression and regression of coronary artery disease in symptomatic men with normal to moderately elevated serum cholesterol levels. The Regression Growth Evaluation Statin Study (REGRESS). Circulation 91, 2528-2540.
12. Kennedy, M. A., Barrera, G. C., Nakamura, K., Baldan, A., Tarr, P., Fishbein, M. C., Frank, J., Francone, O. L., and Edwards, P. A. (2005). ABCG1 has a critical role in mediating cholesterol efflux to HDL and preventing cellular lipid accumulation. Cell Metab 1, 121-131.
13. Kirov, G., Lowry, C. A., Stephens, M., Oldfield, S., O'Donovan, M. C., Lightman, S. L., and Owen, M. J. (2001). Screening ABCG1, the human homologue of the Drosophila white gene, for polymorphisms and association with bipolar affective disorder. Mol Psychiatry 6, 671-677.
14. Langmann, T., Porsch-Ozcurumez, M., Unkelbach, U., Klucken, J., Schmitz, G. Genomic organization and characterization of the promoter of the human ATP-binding cassette transporter-G1 (ABCG1) gene. Biochim. Biophys. Acta 1494: 175-180, 2000.
15. Larrede, S., Quinn, C. M., Jessup, W., Frisdal, E., Olivier, M., Hsieh, V., Kim, M. J., Van Eck, M., Couvert, P., Carrie, A., et al. (2009). Stimulation of cholesterol efflux by LXR agonists in cholesterol-loaded human macrophages is ABCA1-dependent but ABCG1-independent. Arterioscler Thromb Vasc Biol 29, 1930-1936.
16. Le Goff, W., Guerin, M., Nicaud, V., Dachet, C., Luc, G., Arveiler, D., Ruidavets, J. B., Evans, A., Kee, F., Morrison, C., et al. (2002). A novel cholesteryl ester transfer protein promoter polymorphism (−971G/A) associated with plasma high-density lipoprotein cholesterol levels. Interaction with the TaqIB and −629C/A polymorphisms. Atherosclerosis 161, 269-279.
17. Lindqvist, P., Ostlund-Lindqvist, A. M., Witztum, J. L., Steinberg, D., and Little, J. A. (1983). The role of lipoprotein lipase in the metabolism of triglyceride-rich lipoproteins by macrophages. J Biol Chem 258, 9086-9092.
18. Lorkowski, S., Rust, S., Engel, T., Jung, E., Tegelkamp, K., Galinski, E. A., Assmann, G., Cullen, P. Genomic sequence and structure of the human ABCG1 (ABC8) gene. Biochem. Biophys. Res. Commun. 280: 121-131, 2001.
19. Mauldin, J. P., Nagelin, M. H., Wojcik, A. J., Srinivasan, S., Skaflen, M. D., Ayers, C. R., McNamara, C. A., and Hedrick, C. C. (2008). Reduced expression of ATP-binding cassette transporter G1 increases cholesterol accumulation in macrophages of patients with type 2 diabetes mellitus. Circulation 117, 2785-2792.
20. Milosavljevic, D., Kontush, A., Griglio, S., Le Naour, G., Thillet, J., and Chapman, M. J. (2003). VLDL-induced triglyceride accumulation in human macrophages is mediated by modulation of LPL lipolytic activity in the absence of change in LPL mass. Biochim Biophys Acta 1631, 51-60.
21. Nakamura, M., Ueno, S., Sano, A., and Tanabe, H. (1999). Polymorphisms of the human homologue of the Drosophila white gene are associated with mood and panic disorders. Mol Psychiatry 4, 155-162.
22. Souverein, O. W., Jukema, J. W., Boekholdt, S. M., Zwinderman, A. H., and Tanck, M. W. (2005). Polymorphisms in APOA1 and LPL genes are statistically independently associated with fasting TG in men with CAD. Eur J Hum Genet 13, 445-451.
23. Spielmann, N., Mutch, D. M., Rousseau, F., Tores, F., Hager, J., Bertrais, S., Basdevant, A., Tounian, P., Dubern, B., Galan, P., and Clement, K. (2008). Cathepsin S genotypes are associated with Apo-A1 and HDL-cholesterol in lean and obese French populations. Clin Genet 74, 155-163.
24. Stengel, D., Antonucci, M., Gaoua, W., Dachet, C., Lesnik, P., Hourton, D., Ninio, E., Chapman, M. J., and Griglio, S. (1998). Inhibition of LPL expression in human monocyte-derived macrophages is dependent on LDL oxidation state: a key role for lysophosphatidylcholine. Arterioscler Thromb Vasc Biol 18, 1172-1180.
25. Takahashi, S., Suzuki, J., Kohno, M., Oida, K., Tamai, T., Miyabo, S., Yamamoto, T., and Nakai, T. (1995). Enhancement of the binding of triglyceride-rich lipoproteins to the very low density lipoprotein receptor by apolipoprotein E and lipoprotein lipase. J Biol Chem 270, 15747-15754.
26. Thomassen, M. J., Barna, B. P., Malur, A. G., Bonfield, T. L., Farver, C. F., Malur, A., Dalrymple, H., Kavuru, M. S., and Febbraio, M. (2007). ABCG1 is deficient in alveolar macrophages of GM-CSF knockout mice and patients with pulmonary alveolar proteinosis. J Lipid Res 48, 2762-2768.
27. Van Eck, M., Zimmermann, R., Groot, P. H., Zechner, R., and Van Berkel, T. J. (2000). Role of macrophage-derived lipoprotein lipase in lipoprotein metabolism and atherosclerosis. Arterioscler Thromb Vasc Biol 20, E53-62.
28. Wang, N., Lan, D., Chen, W., Matsuura, F., and Tall, A. R. (2004). ATP-binding cassette transporters G1 and G4 mediate cellular cholesterol efflux to high-density lipoproteins. Proc Natl Acad Sci USA 101, 9774-9779.
29. Whitehead K A, Langer R, Anderson D G, Nat Rev Drug Discov. 2009 February; 8(2):129-38, Knocking down barriers: advances in siRNA delivery.
30. Yvan-Charvet, L., Ranalletta, M., Wang, N., Han, S., Terasaka, N., Li, R., Welch, C., and Tall, A. R. (2007). Combined deficiency of ABCA1 and ABCG1 promotes foam cell accumulation and accelerates atherosclerosis in mice. J Clin Invest 117, 3900-3908.

1-15. (canceled)

16. A method for treating obesity in a patient, which method comprises administering an inhibitor of ATP-binding cassette G1 (ABCG1) gene expression or activity to the patient.

17. The method of claim 16, wherein the inhibitor is a nucleic acid.

18. The method of claim 17, wherein the inhibitor is a siRNA.

19. The method of claim 18, wherein the siRNA is in form of a synthetic RNA duplex (ds-siRNA).

20. The method of claim 18, wherein the inhibitor is a siRNA that comprises a nucleotide sequence selected from the group consisting of SEQ ID NO: 1 to SEQ ID NO:8.

21. The method of claim 17, wherein the inhibitor is an antisense nucleic acid.

22. The method of claim 17, wherein the nucleic acid is carried by an expression vector.

23. The method of claim 22, wherein the expression vector is a virus vector, preferably an adenovirus vector or a lentivirus vector.

24. The method of claim 17, wherein the inhibitor is formulated in a nanoparticle.

25. The method of claim 16, wherein obesity is abdominal obesity.

26. The method of claim 16, wherein the inhibitor is administered by subcutaneous, intradermal, or intramuscular injection.

27. The method of claim 25, wherein the inhibitor is administered by abdominal injection in the patient.

28. An in vitro method for determining whether a patient is at risk of developing obesity, which method comprises detecting the presence of a mutation, substitution or deletion of at least one nucleotide in ABCG1 gene or regulatory sequences thereof.

29. The method of claim 28, which comprises detecting the presence of a nucleotide substitution in the promoter of ABCG1 gene.

30. The method of claim 28, wherein the nucleotide substitution is at position −134 or −204 from the starting codon of the ABCG1 gene (respectively designated as single nucleotide polymorphism rs1378577 and rs1893590), wherein the presence of a T a position −134 and/or a A at position −204 is indicative of a risk of developing obesity.
